(19) United States
(12) Patent Application Publication
(10) Pub. No.: US 2021/0195857 A1
(43) Pub. Date: Jul. 1, 2021

(54) AN UNDERGROUND SOIL SENSORS SYSTEM

(71) Applicant: CropX Technologies Ltd., Herzliya (IL)

(72) Inventor: Yossi Haran, Modi'in-Macabim-Reut (IL)

(21) Appl. No.: 16/071,210

(22) PCT Filed: Jan. 19, 2017
(86) PCT No.: PCT/IL2017/050082

§ 371 Date: Jul. 19, 2018

Related U.S. Application Data
(60) Provisional application No. 62/280,914, filed on Jan. 20, 2016.

(51) Int. Cl. A01G 25/16; G01N 33/24; G08C 17/02

(52) U.S. Cl. CPC A01G 25/167; G01N 2033/245; G08C 17/02; G01N 33/246

(57) ABSTRACT

An underground soil sensors system is disclosed. The underground soil sensors system can include a base station having at least one antenna. The underground soil sensors system can include at least one set of soil sensors, where each soil sensor in the at least one set can be positioned at a predetermined vertical distance below a surface of a target soil. Each soil sensor in the at least one set can transmit signals to an adjacent soil sensor positioned thereabove in that set and to receive signals from an adjacent soil sensor positioned therebelow in that set. A topmost soil sensor in the at least one set can transmit signals to the at least one antenna of the base station.

### BACKGROUND OF THE INVENTION

### 1. Technical Field

The present invention relates to the field of soil sensors, and more particularly, to an underground soil sensors system.

### 2. Discussion of Related Art

Current volumetric water content (VWC) profile sensors can include a pole and/or circular radiofrequency (RF) electrodes wrapped around the pole. Typically, current VWC sensors can significantly disturb a target soil during an installation and/or can require pre-drilling procedures in order to be installed. Unmatched pre-drilling and VWC sensor's dimensions can result in a poor contact between the VWC sensor and the soil. The poor contact between the soil sensor and the soil and/or disturbed soil can introduce measurement errors. For example, a gap can be generated between the VWC sensor and the soil, in which vertical water flow and/or accommodation can occur, thereby affecting the VWC measurement of the target soil. Moreover, the pre-drilling requirement can increase the installation costs.

Current solutions for transmitting radiofrequency (RF) signals from soil sensors to a base station can include wired and/or wireless connections. Wired connections between the soil sensors and/or between the soil sensors and the base station can be damaged during a working of a target soil. Moreover, wired connections can increase installation costs. Wireless connections between the soil sensors and the base station can be restricted by a reduced transmitting power of the soil sensors due to soil attenuation of the RF signals. Accordingly, a depth at which the soil sensors can be installed can be limited.

### SUMMARY OF THE INVENTION

One aspect of the present invention provides a soil sensor assembly including: a rotatably anchorable portion to be rotatably anchored in a soil; at least one soil sensor mounted onto the rotatably anchorable portion; and a communicator for communicating at least one output of the at least one soil sensor to a location remote from the at least one soil sensor assembly.

Another aspect of the present invention provides a volumetric water content (VWC) sensor including: a support to enable installation of the VWC sensor in a target soil; at least one VWC probe positioned at a predefined longitudinal location along the support, the at least one VWC probe including: a helical blade secured along its inner lateral side to an outer surface of the support, and at least one radiofrequency (RF) electrode secured to the helical blade at a predefined radial distance from the support; and at least one electronics unit coupled to the at least one RF electrode to transmit and receive RF signals from the at least one RF electrode.

Another aspect of the present invention provides a volumetric water content (VWC) sensor comprising: at least one VWC probe including at least two radiofrequency (RF) electrodes, the at least one VWC probe to measure a VWC of a target soil in a measurement region between the at least two RF electrodes, and a support to secure positioning of the at one VWC probe, wherein the support occupies less than 10% of the measurement region.

Another aspect of the present invention provides an underground soil sensors system, the system comprising: a base station comprising at least one antenna; and at least one set of soil sensors, each soil sensor in the at least one set is positioned at a predetermined vertical distance below a surface of a target soil, wherein each soil sensor in the at least one set to transmit signals to an adjacent soil sensor positioned thereabove in that set and to receive signals from an adjacent soil sensor positioned therebelow in that set, and wherein a topmost soil sensor in the at least one set to transmit signals to the at least one antenna of the base station.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1A is an illustration of a underground soil sensors system and its undisturbed soil installation, according to some embodiments of the invention (on a left-hand side of FIG. 1A) and current soil sensors and installation methods of slurry installation of a profiling sensor, dug installation of scientific sensors and trench installation of scientific sensor, according to the prior art.

FIGS. 1B-1C are illustrations of a volumetric water content (VWC) sensor, according to some embodiments of the invention;

FIGS. 2A-2C are illustrations of disassembled volumetric water content (VWC) sensor, according to some embodiments of the invention;

FIGS. 3A-3B are illustrations of various configurations of a tip of volumetric water content (VWC) sensor, according to some embodiments of the invention;

FIGS. 4A-4D are illustrations of various configurations of radiofrequency (RF) electrodes of a volumetric water content (VWC) sensor, according to some embodiments of the invention;

FIGS. 5A-5E are illustrations of a volumetric water content (VWC) sensor including radiofrequency (RF) electrodes protruding above at least one surface of helical blades, according to some embodiments of the invention.

FIG. 6 is an illustration of configuration of volumetric water content (VWC) sensor with a support being a coreless helical blade, according to some embodiments of the invention;

FIG. 7 is an illustration of a volumetric water content (VWC) probe including segmented RF electrodes, according to some embodiments of the invention;

FIG. 8 is a schematic block diagram illustrating an electronics unit of volumetric water content (VWC) sensor, according to some embodiments of the invention;

FIG. 9 is a schematic block diagram of an electronic circuitry of electronics unit of volumetric water content (VWC) sensor, according to some embodiments of the invention;

FIG. 10 is a flowchart illustrating a method of measuring a undisturbed volumetric water content (VWC), according to some embodiments of the invention;

FIG. 11 is a flowchart illustrating a method of installing a soil sensor assembly, according to some embodiments of the invention;

FIG. 12A is a graph illustrating volumetric water content (VWC) measurement results being measured by a prior art profile sensor, according to the prior art;

FIG. 12B is a graph illustrating volumetric water content (VWC) measurement results being measured by a VWC sensor, according to some embodiments of the invention;

FIGS. 13A-13C are illustrations of an underground soil sensors system, according to some embodiments of the invention;

FIGS. 13D-13E are illustrations of a set of soil sensors, according to some embodiments of the invention;

FIG. 13F is an illustration of an inverse ground-penetrating radar (IGPR) tool in a topmost sensor in set of underground soil sensors system, according to some embodiments of the invention;

FIG. 14A is an illustration of a soil sensor, according to some embodiments of the invention;

FIG. 14B is an illustration of a cross-section of an installing tool interface of a soil sensor, according to some embodiments of the invention;

FIG. 14C-14E are illustrations of an installing tool for a soil sensor, according to some embodiments of the invention; and

FIG. 15 is a flowchart illustrating a method of determining a profile of properties of a target soil, according to some embodiments of the invention.

### DETAILED DESCRIPTION OF THE INVENTION

Effective agriculture can depend on obtaining accurate, continuous, in-field soil measurements, for example soil moisture measurements, including soil measurements at different sub-surface depths. A target soil can be non-uniform and therefore continuous measurements can be required to be measured at multiple locations in a field to, for example, best inform agricultural actions. For example, different parts of the field can require different amounts of irrigation, which can require continuous soil-moisture monitoring at different specific locations in the field. Current soil sensor devices can invariably provide biased measurements of sub-soil due to the disturbance of the soil, caused by, for example, their installation. Current scientific installation procedures that can provide, for example, unbiased measurements, can be complex and/or impractical in a working agricultural field. Current soil sensor devices typically do not provide practical, accurate, continuous and/or in-field soil measurements of sub-surface soil. The present invention describes a soil sensor device, which can provide continuous, unbiased measurement of un-disturbed sub-surface soil, and/or can include a simple do-it-yourself installation.

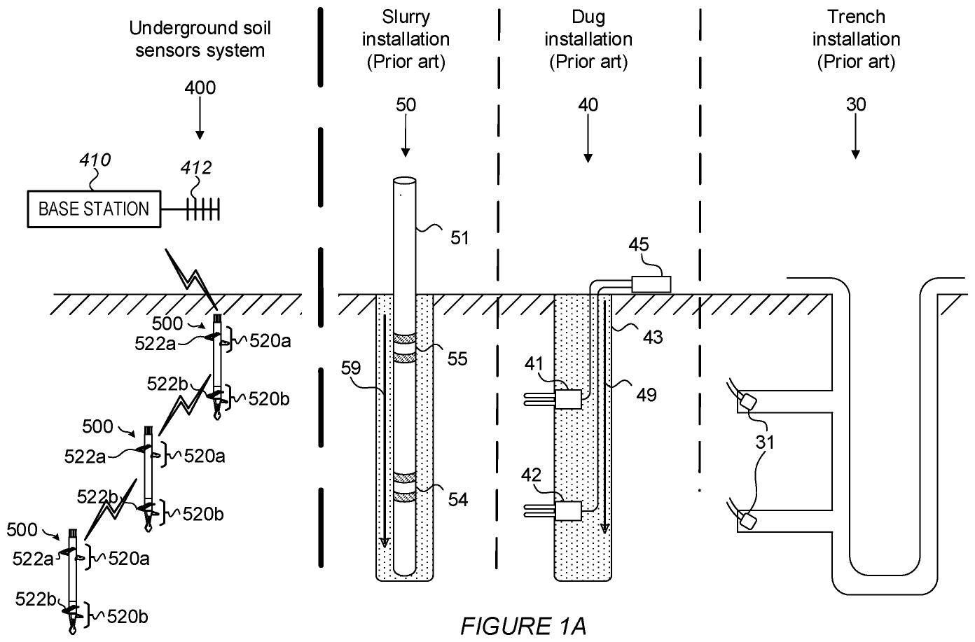

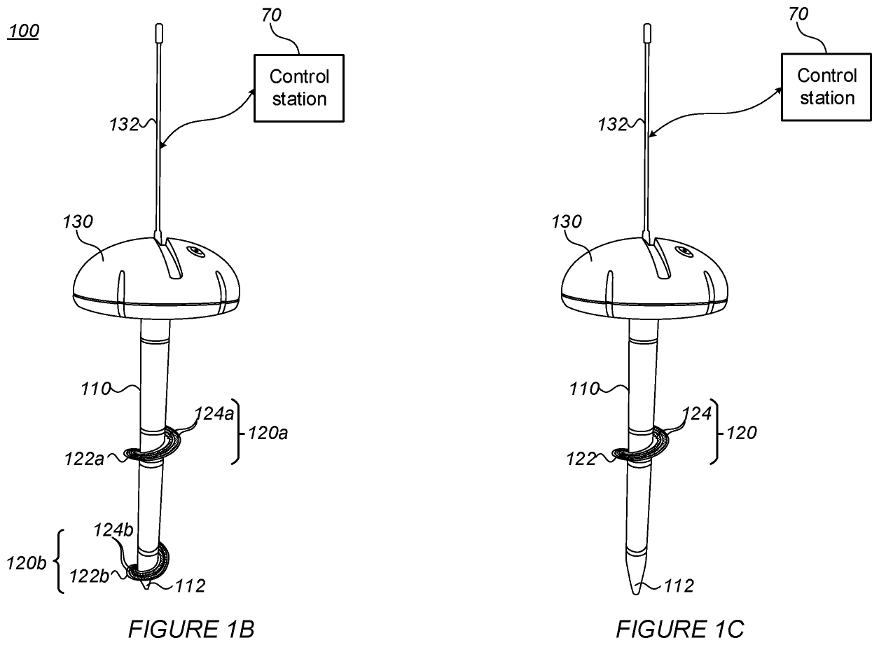

FIG. 1A presents an underground soil sensors system 400 and its undisturbed soil installation, according to some embodiments of the invention (on a left-hand side of FIG. 1A) and on a right-hand side of FIG. 1A, current soil sensors and installation methods of slurry installation 50 of a profiling sensor, dug installation 40 of scientific sensors and trench installation 30 of scientific sensor, according to the prior art.

Slurry installation 50 can typically include drilling a wide-bore vertical hole, preparing slurry by mixing the soil from the hole with water, pouring the slurry back into the hole, and/or placing a pole-shaped profiling sensor 51 into the into slurry-filled vertical hole. The profiling sensor 51 can be therefore in contact with slurry 52, ensuring close contact of top sensor 53 and bottom sensor 54 with the slurried soil. One disadvantage of slurry installation can be that slurry 52 is a disturbed soil medium, which can enhance a vertical flow 59 of water through the slurry 52, thereby biasing the measurements of the sensors 55, 54. For example, measurements of bottom sensor 54 can be prone to reflect soil moisture that can be actually that of top soil due to, for example, excessive vertical flow 59 of water, through the slurry 52. Typically, following an irrigation event, measurements from the bottom sensor 54 can erroneously show a rise in soil moisture that can be similar in timing and amplitude, to measurements of the top sensor 55. Such measurements can be biased, since water takes time to filtrate down through undisturbed soil, as is well known in the art.

Dug installation can include a vertical hole being dug, through which the sensors, e.g., top scientific sensor 41 and bottom scientific sensor 42, can be placed at different desired depths, such that their sensing part, e.g., prongs, are pierced into the wall of the hole to measure intact soil. The sensors 41 and 42 can be typically connected by wire to a logger 45 on the ground, and the hole is then filled with soil-fill 43. One disadvantage of dug installation method can include disturbed soil-fill 43, through which a vertical flow 49 of water can occur. Thereby, bottom scientific sensor 42 can give erroneous measurements that correspond in timing and amplitude to those of top scientific sensor 41, reflecting unnaturally excessive vertical flow 49 of water through the soil fill 43. Another disadvantage of dug installation 40 can include difficult and time-consuming installation.

Trench installation 30 can provide a scientifically robust method for installing scientific sensors 31. One disadvantage of the trench installation can include impractical implementation in an active agricultural field. In this method, a deep trench, trench installation can typically include drilling one yard deep and wide, dug and/or wide bore (e.g., 60 cm) vertical peers into a wall of the trench at the desired depths, and/or manually placing scientific sensors through the vertical peers, and/or piercing their sensing prongs into undisturbed soil at the far end of the peer, at an upward angle of 45 degrees, so that no seepage (or substantially no seepage) of water through the peer to affect the sensor prongs can occur. The trench can be covered with a tarp to, for example, prevent accumulating water to enter the peers, and/or accumulated water to be pumped from the tarp covered trench. Trench installation 30 can avoid bias of disturbed soil and vertical flow, however—it can be utterly impractical in the setting of an agricultural field. More so when multiple measurements are needed from different parts of a field.

Currently available soil sensor devices can provide biased measurements, due to, for example, measuring disturbed soil, and/or due to, for example, biased vertical water flow. While the description above is of soil moisture measurements, the same can be true for other measurements that can include soil nutrients, micro nutrients, genetic measurements, organic compounds, and many other measurements.

Generally, the present invention includes an underground soil sensors system 400 that can include soil sensors 500, which can be installed into sub-surface soil, and/or can provide unbiased measurements from undisturbed soil. In some embodiments, soil sensor 500 is rotatably anchorable soil sensor. Soil sensor 500 can include soil probes 520 at multiple depths, such as a top soil probe 520a located on, or integrated into a helical blade 522a, and/or a soil probe 520b located on or integrated helical blade 522b. Soil sensor 500 can be installed by rotating it into the subsurface soil, and so both helical blades 522a, 522b can be cut into the subsurface soil, thereby placing soil probes 520a, 520b in direct contact with undisturbed soil, and/or providing unbiased measurements from the soil, measurements that may not be subject to excessive vertical water flow. Installation of soil sensor 500 can eliminate the necessity for a slurry and/or soil-fill, thereby eliminating (or substantially eliminating) bias measurements due to, for example, vertical flow. Soil probes 520a, 520b can include helical blades 522a, 522b away from pole 510 of the spiral sensor 500, and can minimize possible vertical flow along shaft 510 to bias the readings of the sensor. Spiral sensor 500 can provide accurate soil measurements of undisturbed soil and/or provide measurement that are not unbiased by artifactual vertical flow.

Advantageously, disclosed soil sensor(s) 500 can provide measurement results without disturbing the soil. In some embodiments, spiral sensor(s) 500 can be installed in a simple manner and can use five to ten fold shorter installation time with respect to the prior art, for example in the order of magnitude of minutes or tens of minutes instead of hours. Advantageously, in some embodiments, disclosed spiral sensor(s) 500 may revolutionize the domain of soil sensors, offering for the first time, a device that provides continuous, accurate, soil measurements of undisturbed soil, unbiased by inadvertent vertical water flow, and with an unprecedented simplicity and speed of a truly do-it-yourself installation.

Underground soil sensors system 400 can include a base station 410 having at least one antenna 412. Underground soil sensors system 400 can include at least one set of soil sensors (for example, soil sensors 500). Each soil sensor in the at least one set can be positioned at a predetermined vertical distance below a surface of a target soil. Each soil sensor in the at least one set can transmit signals to an adjacent soil sensor positioned thereabove in that set and to receive signals from an adjacent soil sensor positioned therebelow in that set. A topmost soil sensor in the at least one set can transmit signals to the at least one antenna 412 of the base station 410.

A soil sensor assembly and methods of measuring undisturbed soil are disclosed. The soil sensor assembly can be a volumetric water content (VWC) sensor. The soil sensor assembly can include at least one soil probe. The soil probes can be secured to a support to enable an installation of the soil sensor assembly in a target soil. The soil probes can include helical blades secured concentrically along the support at predefined longitudinal locations. The soil probes can include at least one radiofrequency (RF) electrode secured to the helical blades at a predefined radial distance from a longitudinal axis of the support. The soil sensor assembly can also include at least one electronics unit coupled to the RF electrodes to receive and/or transmit RF signals from the RF electrodes. The soil sensor assembly can enable a self-tapping installation action and/or enable alienating the soil measurements (e.g., by RF electrodes) away from a disturbed soil. The soil sensor assembly can enable measuring properties of undisturbed soil and/or eliminate a vertical water flow along the sensor thereof.

FIGS. 1B-1C are illustrations of a volumetric water content (VWC) sensor 100, according to some embodiments of the invention. VWC sensor 100 can include a support 110. In some embodiments, support 110 is a rotatably anchorabable portion. In some embodiments, support 110 is a pole (e.g., as illustrated in FIGS. 1B-1C). In various embodiments, pole 110 is a monolith having a tapered nail-like shape and/or includes a tip 112. Tip 112 can have a tapered shape that can enable initial penetration of VWC sensor 100 into a target soil during an installation process.

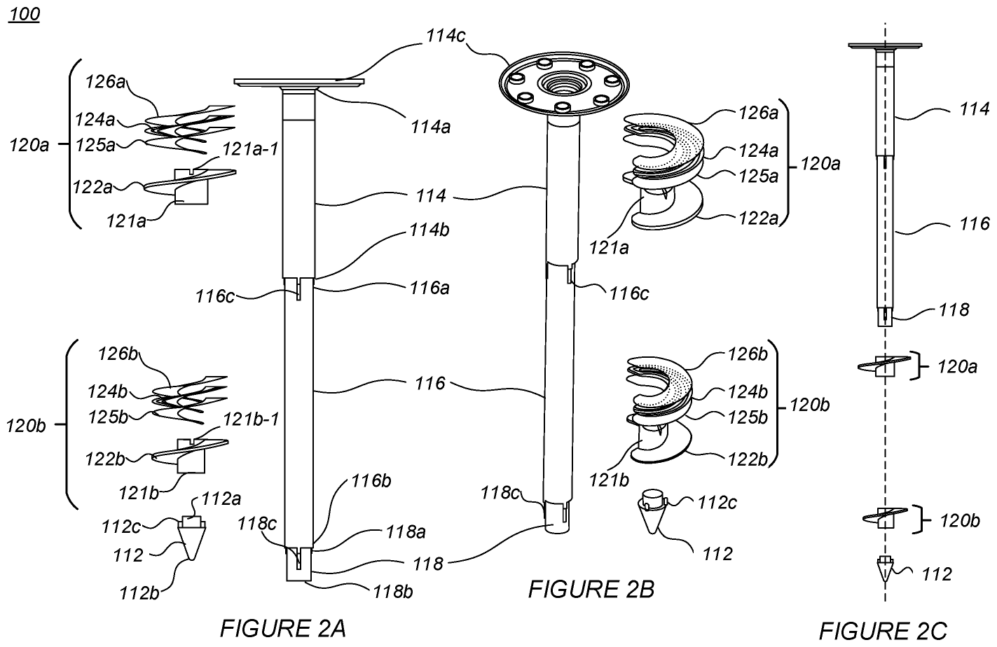

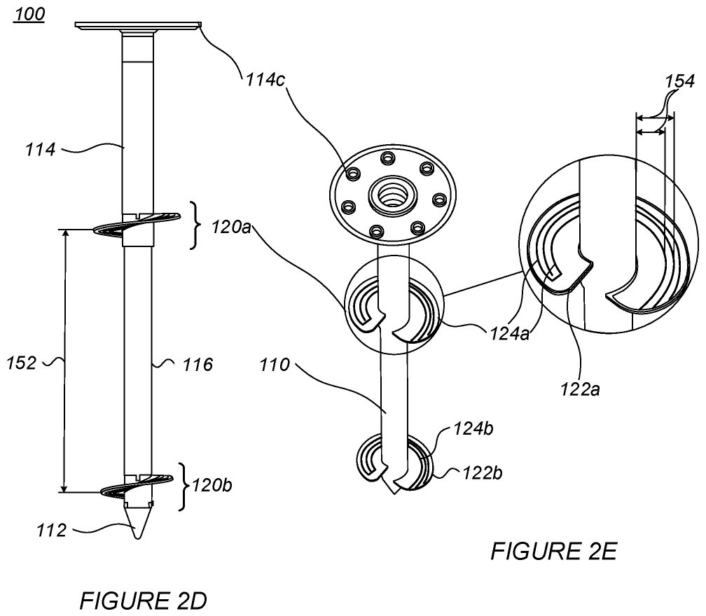

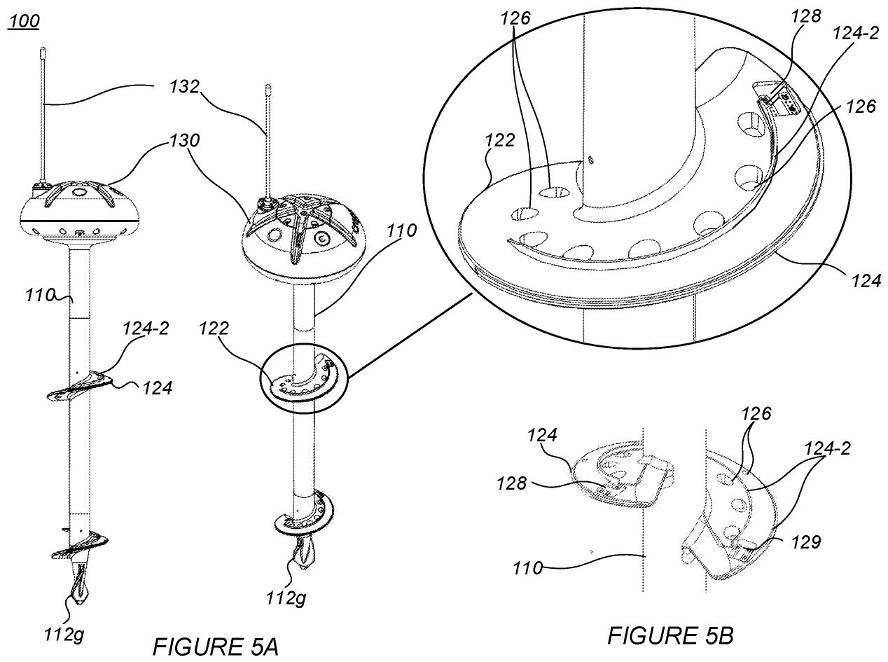

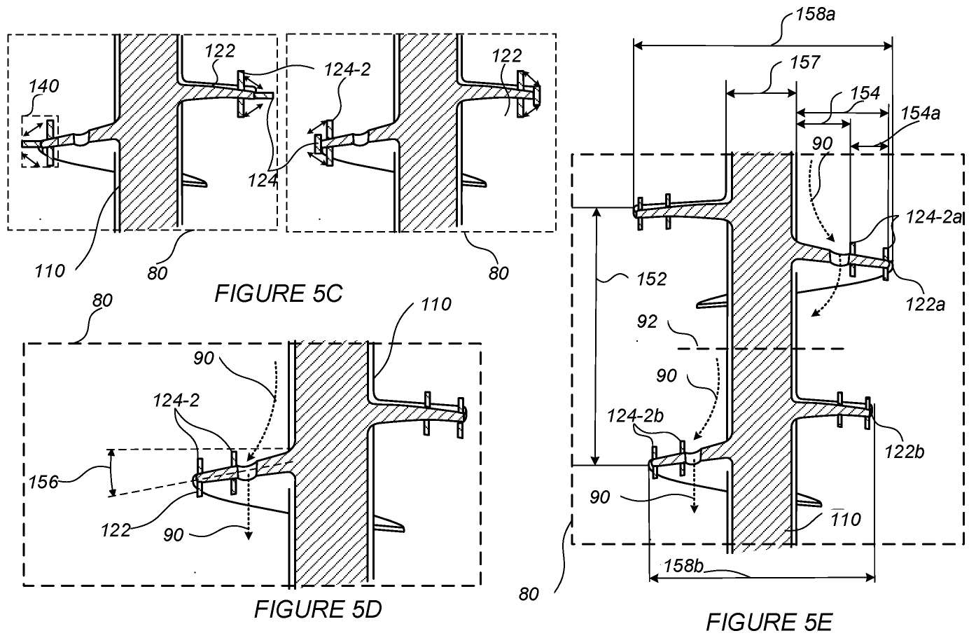

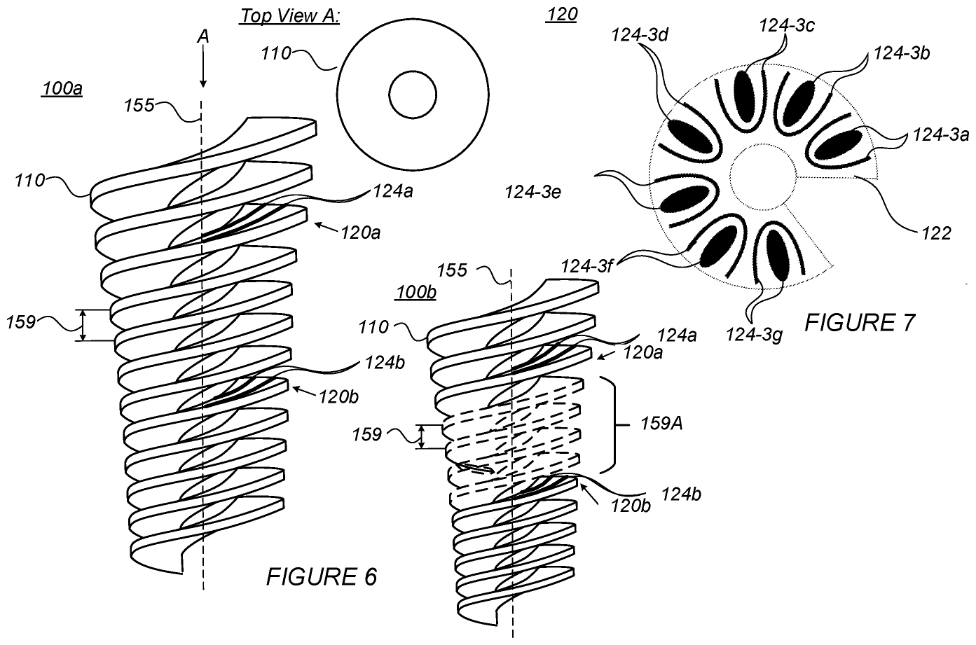

VWC sensor 100 can include at least one VWC probe 120 secured to an outer surface of pole 110 at predefined longitudinal location along the pole (e.g., as described in detail with respect to FIGS. 5-6). In some embodiments, VWC sensor 100 includes single VWC probe 120, as shown in FIG. 1B. In some embodiments, VWC sensor 100 includes two VWC probes 120a, 120b separated by a longitudinal distance 152 (e.g., as shown in FIG. 1B, FIG. 2D) that can enable measuring VWC of a target soil at two depths (e.g., profile VWC sensor). In some embodiments, the two depths are different. In various embodiments, VWC sensor 100 also includes at least one additional soil sensor, for example, a temperature sensor, a pH sensor, a pressure sensor, a salinity sensor and/or sensor for determining level of minerals in a target soil.

In some embodiments, each of VWC probes 120 (e.g., each of VWC probes 120a, 120b as shown in FIG. 1B) includes a helical blade 122 secured along an inner lateral side to an outer surface of pole 110. Helical blade 122 can complete a helical path of at least 360° around pole 110. In some embodiments, helical blade 122 can complete 720° around pole 110. Helical blade 122 can enable performing a screwing motion of VWC sensor 100 within a target soil during an installation process.

VWC probe 120 (e.g., each of VWC probes 120a, 120b as shown in FIG. 1B) can include radiofrequency (RF) electrodes 124a and 124b, generally electrodes 124, secured to helical blade 122 at a predefined radial distance 154 from pole 110 (e.g., as shown in FIG. 2E). RF electrodes 124 can have a helical shape that corresponds to shape of helical blade 122 and/or can complete a helical path of at least 360° around pole 110. RF electrodes 124 can be surface electrodes and/or can be secured to at least one of surfaces of helical blade 122. In some embodiments, RF electrodes 124 can be embedded within helical blade 122. RF electrodes 124 can cover at least a portion of the surfaces thereof. An RF field can be generated by adjacent RF electrodes 124 to measure a VWC of a target soil in a measurement region between the adjacent RF electrodes. In some embodiments, the entirety of the helical blade 122 is a RF electrode.

The predefined radial distance 154 can be predefined based on a desired RF field to be generated by RF electrodes 124 and/or to alienate RF electrodes 124 from pole 110 and/or from a disturbed target soil. In some embodiments, RF electrodes 124 are positioned at 30% most lateral portion of helical blades 122.

During a screwing motion of an installation process, helical blade 122b of VWC probe 120b can enter an undisturbed target soil, thereby providing a good contact between helical blade 122b and/or RF electrodes 124b and the target soil. Longitudinal distance 152 between helical blades 122a, 122b and/or diameters of helical blades 122a, 122b can be predefined to, for example, optimize the accuracy of VWC measurement of the target soil and/or to provide a good contact between helical blade 122a and/or RF electrodes 124a and the target soil. For example, a diameter of helical blade 122a can be greater than a diameter of helical blade 122b (e.g., as shown in FIG. 1B) such that helical blade 122a, which can follow a screwing path of helical blade 122b during the screwing motion of the installation process, enters a undisturbed soil, thereby providing a good contact between helical blade 122a and/or RF electrodes 124a and a target soil.

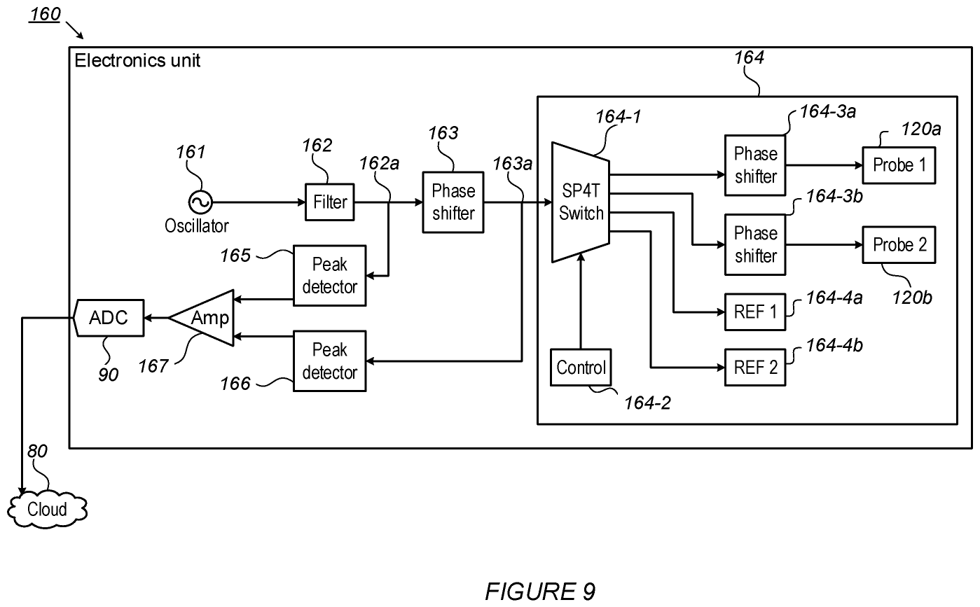

VWC sensor 100 can include at least one electronics unit (e.g., electronics unit 160 as shown in FIGS. 8-9) that can transmit and/or receive RF signals from RF electrodes 124. In some embodiments, at least one of the electronics units is at least partially embedded within pole 110. In some embodiments, at least one of the electronics units is at least partially embedded within helical blade 122 of at least one of VWC probes 120. In some embodiments, VWC sensor 100 includes an electronics bay 130 secured to pole 110 at the end being opposite to tip 112. Electronics bay 130 can include at least one of the electronics units. RF electrodes 124 of VWC probe 120 can be connected to the electronic units and/or to electronics bay 130 using wiring and/or wireless connections (not shown). In some embodiments, electronics bay 130 includes an antenna 132. In some embodiments, electronics units and/or electronics bay 130 include a wireless communications device (e.g., wireless communicator) that can enable transmitting the received RF signals (e.g., by antenna 132) to a remote control station 70. The wireless communications device can be any wireless communications device as is known in the art.

FIGS. 2A-2C are illustrations of disassembled volumetric water content (VWC) sensor 100, according to some embodiments of the invention. FIGS. 2D-2E are illustrations of assembled VWC sensor 100, according to some embodiments of the invention. FIGS. 2A, 2C, 2D provide a side view and FIGS. 2B, 2E provide an isometric view of VWC sensor 100.

In some embodiments, pole 110 of VWC sensor 100 includes a first tubular section 114, a second tubular section 116 and/or a third tubular section 118. First tubular section 114 can have a first end 114a and a second end 114b, second tubular section 116 can have a first end 116a and a second end 116b and/or third tubular section 118 can have a first end 118a and a second end 118b.

In some embodiments, first end 114a of first tubular section 114 includes connector 114c that can connect electronics bay 130 to pole 110. Connector 114c can include any connection means known in the art. In some embodiments, second tubular section 116 proceeds coaxially from second end 114b of first tubular section 114 and/or third tubular 118 section tubular section proceeds coaxially from second end 116b of second section 116. Diameters and lengths of first tubular section 114, second tubular section 116 and/or third tubular section 118 can be predefined to provide a tapered shape for pole 110. For example, as shown in FIGS. 2A-2E, diameter of second tubular section 116 can be smaller than diameter of first tubular section 114 and/or diameter of third tubular section 118 can be smaller than diameter of second tubular section 116. In various embodiments, first tubular section 114 has diameter of 30 mm and/or length of 177 mm, second tubular section 116 has diameter of 26 mm and/or length of 250 mm and/or third tubular section 118 has diameter of 20 mm. In various embodiments, pole 110 and/or each of tubular sections 114, 116 and/or 118 include a screw-thread to enhance a screw motion of VWC sensor 100 during the installation process.

In various embodiments, first end 116a of second tubular section 116 includes connectors 116c and/or first end 118a of third tubular section 118 includes connectors 118c. Connectors 116c, 118c can be protrusions and/or can be located equally about an outer surface of pole 110 (e.g., as shown in FIGS. 2A-2C).

In some embodiments, VWC sensor 100 includes a first VWC probe 120a and a second VWC probe 120b. Helical blade 122a of first VWC probe 120a can be connected to an outer surface of a cylindrical shell 121a and/or helical blade 122b of second VWC probe 120b can be connected to an outer surface of a cylindrical shell 121b. Cylindrical shells 121a, 121b can have diameters that match the diameters of second and third tubular sections 116, 118, respectively. Cylindrical shells 121a, 121b can also include matching connectors 121a-1, 121b-1 (e.g., indents as illustrated in FIGS. 2A-2C) that can be connected to connectors 116c, 118c and can secure first and second VWC probes 120a, 120b to pole 110.

In some embodiments, tip 112 of VWC sensor 100 has a first end 112a and a second end 112b. First end 112a can have a diameter that match the diameter of second end 118b of third tubular section 118. First end 112a of tip 112 can also include connectors 112c (e.g., protrusions as shown in FIGS. 2A-2C) and/or shell 121b of second VWC probe 120b can include matching connectors 118d (e.g., indents as shown in FIGS. 2A-2C) such that tip 112 can be connected and/or secured to third tubular section 118 and/or to shell 121b of second VWC probe 120b. In some embodiments, second end 112b of tip 112 has a tapered shape that can allow for, for example, VWC sensor 100 to penetrate to a target soil during an installation procedure.

In some embodiments, connectors 112c, 116c, 118c and/or 118d include catches know in the art (e.g., detents) that can enhance securing of VWC probes 120 and tip 112 to pole 110.

The diameter of first VWC probe 120a that can match the diameter of second tubular section 116, the diameter of second VWC probe 120b that can match the diameter of third tubular section 118 and/or the diameter of first end 112a of tip 112 that can match the diameter of second end 118b of third tubular section 118 can simplify the assembly of VWC sensor 100, as shown in FIG. 2C. The assembled VWC sensor 100 is shown in FIGS. 2D-2E.

In some embodiments, VWC probe 120 includes three layers: a first layer that includes helical blade 122, a second layer that includes RF electrodes 124 secured to a substrate 125, and a third protective layer 126 (e.g., as shown in FIGS. 2A-2B). Substrate 125 can be secured to helical blade 122. Protective layer 126 can cover RF electrodes 124 to provide a protection during an installation of VWC sensor 100 within a target soil. In some embodiments, RF electrodes 124 are secured to helical blade 122 (without substrate 125). In some embodiments, helical blade 122 of VWC probe 120 completes a helical path of at least 360° around pole 110. In some embodiments, RF electrodes 124 have a helical shape that corresponds to shape of helical blade 122 and/or complete a helical path of at least 360° around pole 110.

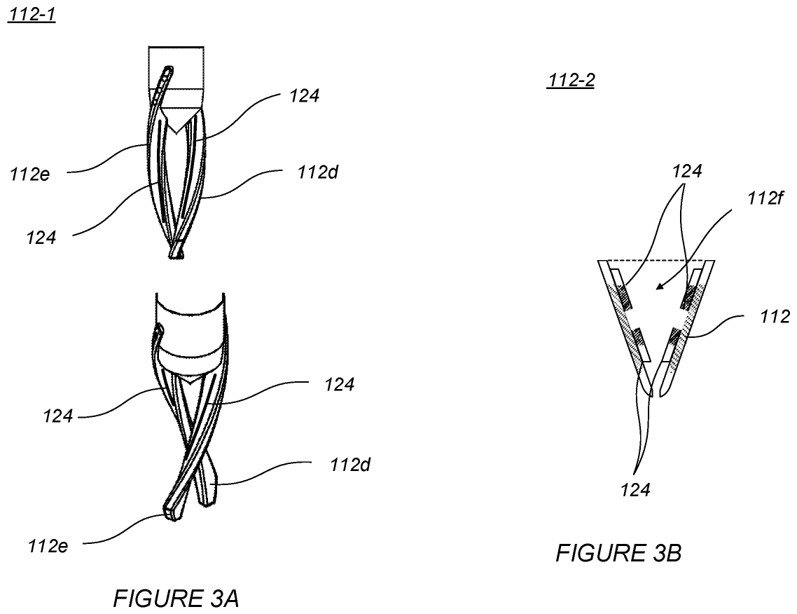

FIGS. 3A-3B are illustrations of various configurations of a tip 112 of a volumetric water content (VWC) sensor 100, according to some embodiments of the invention. FIG. 3A presents an isometric view and a side view of a tip 112-1. FIG. 3B present a cross-section view of tip 112-2.

In some embodiments, tip 112 includes at least two prongs 112d, 112e, where each prong 112d, 112e includes RF electrodes 124 (e.g., as shown in FIG. 3A). Each prong 112d, 112e can have a helical shape and/or can include a nonconductive material. An RF field can be generated by RF electrodes 124 of each prong 122d, 122e to measure a VWC of a target soil in a measurement region between the RF electrodes.

In some embodiments, tip 112 has a gap 112f (e.g., as shown in FIG. 3B). Tip 112 can have a tapered end (e.g., tip 112-2 as shown in FIG. 3A). Tip 112-2 can include RF electrodes 124 secured to an inner lateral surface of the tip within gap 112f.

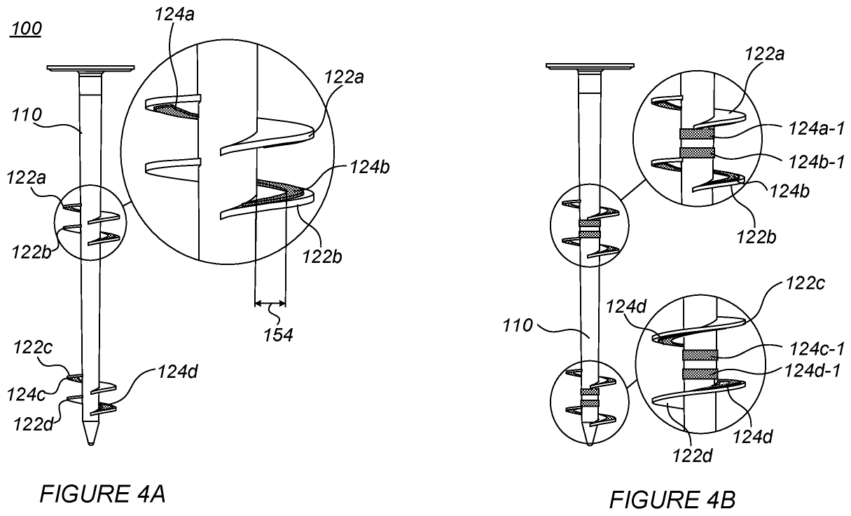

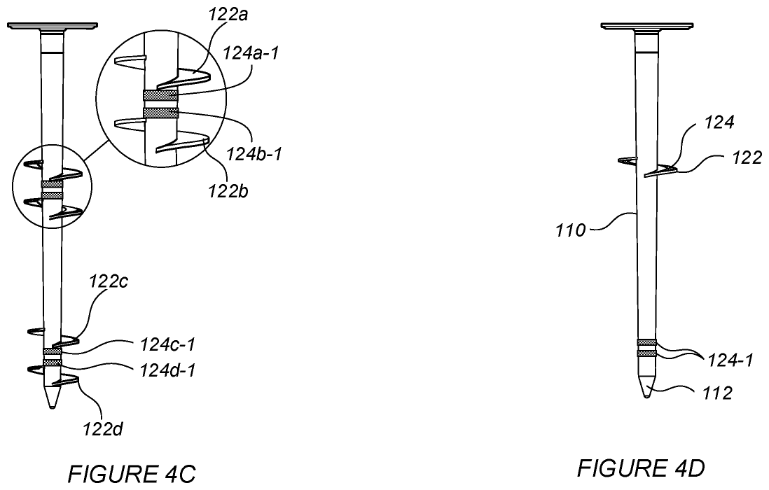

FIGS. 4A-4D are illustrations of various configurations of radiofrequency (RF) electrodes 124 of a volumetric water content (VWC) sensor 100, according to some embodiments of the invention. In some embodiments, two VWC probes 120a, 120b are positioned adjacently at a first predefined longitudinal location along pole 110 and/or two VWC probes 120c, 120d are positioned adjacently at a second predefined longitudinal location along pole 110 (see e.g., FIG. 4A). RF electrodes 124a, 124b of adjacent VWC probes 120a, 120b and/or RF electrodes 124c, 124d of adjacent VWC probes 120c, 120d can face each other. A RF field can be generated by facing RF electrodes 124a, 124b and/or facing RF electrodes 124c, 124d to measure a VWC of a target soil in measurement regions between the RF electrodes. A longitudinal distance between adjacent VWC probes 120a, 120b, a longitudinal distance between adjacent VWC probes 120c, 120d, the first longitudinal location and/or the second longitudinal location can be predefined to, for example, optimize the accuracy of moisture measurements of a target soil and/or to improve a contact between helical blades 122a, 122b, 122cb 122d and the target soil during a screwing motion of an installation process, as described above with respect to FIGS. 5-6.

In some embodiments, VWC sensor 100 includes RF electrodes 124-1. RF electrodes 124-1 can be circular and/or can be secured to an outer surface of pole 110. RF electrodes 124-1 can be surface electrodes. In some embodiments, RF electrodes 124a-1, 124b-1 are positioned between two adjacent VWC probes 120a, 120b and/or RF electrodes 124c-1, 124d-1 are positioned between two adjacent VWC probes 120c, 120d at predefined longitudinal locations (e.g., as shown in FIG. 4B). In some embodiments, RF electrodes 124a-1, 124b-1 and/or RF electrodes 124c-1, 124d-1 are electrical continuations of respective VWC probes 120a, 120b and/or 120c, 120d. In some embodiments, RF electrodes 124-1 only are secured to pole 110 (without RF electrodes 124 secured to helical blades 122). For example, RF electrodes 124-1a, 124-1b, 124-1c, 124-1d as shown in FIG. 4C. In some embodiments, VWC sensor 100 includes at least one VWC probe 120 and/or RF electrodes 124-1, where VWC probe 120 can also include RF electrodes 124, as shown in FIG. 4D.

FIGS. 5A-5E are illustrations of a volumetric water content (VWC) sensor 100 including radiofrequency (RF) electrodes 124-2 protruding above at least one surface of helical blades 122, according to some embodiments of the invention. FIG. 5A present a side view and an isometric view of VWC sensor 100 (a left hand-side and a right-hand side, respectively). FIG. 5B presents an isometric blow-up view of VWC probe 120 of VWC sensor 100. FIGS. 5C-5E present a cross-section view of a portion of VWC sensor 100.

In some embodiments, tip 112 of VWC sensor 100 includes a helical blade 112g (e.g., as shown in FIG. 5A). In some embodiments, RF electrode 124 is secured to an outer lateral side of helical blade 122 of at least one VWC probe 120 (e.g., as shown in FIG. 5A). In some embodiments, at least one RF electrode 124-2 is embedded within helical blade 122 at a predefined radial distance from pole 110 such that embedded RF electrodes 124-2 protrude above at least one of surfaces of helical blade 122 (e.g., as shown in FIGS. 5A-5B). RF electrodes 124-2 can be three-dimensional electrodes and/or can have a helical shape that corresponds to shape of helical blade 122. An RF field can be generated by RF electrodes 124-2 and/or RF electrodes 124 to measure a VWC of a target soil 80 in a measurement region 140 between the RF electrodes, as schematically illustrated by arrows in FIG. 5C. In some embodiments, helical blade 122 and/or pole 110 occupies less than 10% of measurement region 140.

In some embodiments, at least one electronic electrodes interface 128 is embedded within helical blade 122 of VWC probe 120, for example as shown in FIG. 5A. In various embodiments, RF electrodes, for example RF electrodes 124, 124-2, are electrically connected to electronic electrodes interface 128. In some embodiments, a temperature sensor is embedded within electronic electrodes interface 128. The Temperature sensor can include a thermal resistor and/or can measure a temperature of a target soil. The thermal resistor of the temperature sensor can be a part of electrical circuitry of electronic electrodes interface 128 and/or of electronics unit (e.g., electronics unit 160 shown in FIGS. 8-9) and/or can transmit information regarding the temperature by, for example, changing a DC level of RF signals generated by RF electrodes 124 and/or RF electrodes 124-2. In some embodiments, a plurality of sensors are embedded and/or secured to helical blades 122 of VWC probes 120, for example, a pH sensor, a pressure sensor, a salinity sensor and/or a sensor that can measure level of mineral in a target soil.

In various embodiments, two electronic electrodes interfaces 128, 129 are embedded within helical blade 122 of VWC probe 120, as shown for example in FIG. 5B. Electronic electrodes interfaces 128, 129 can be embedded at opposite ends of helical blade 122 such that opposite ends of RF electrodes 124, 124-2 are connected to opposite electronic electrodes interfaces 128, 129 (e.g., as shown in FIG. 5B). Using two electronic electrodes interfaces 128, 129 connected to opposite ends of RF electrodes 124, 124-2 can allow enablement of the electronics unit (e.g., electronics unit 160) to a transmission line based on, for example, time domain transmission (TDT) electronic circuit.

In various embodiments, helical blades 122 include a plurality of holes 126 positioned between pole 110 and protruding RF electrodes 124-2 and/or RF electrodes 124 (e.g., as shown in FIG. 5A-5B). Holes 126 can drain a water 90 flowing along pole 110 and/or along helical blades 122 (e.g., as indicated by dashed arrows in FIG. 5D) to prevent accommodation of the water in a vicinity of RF electrodes 124-2 and/or RF electrodes 124 (e.g., as shown in FIG. 5D). In some embodiments, helical blades 122 are secured to pole 110 at an angle 156 with respect to the pole to provide a slope that facilitates drainage of flowing water 90 (e.g., as shown in FIG. 5D).

In various embodiments, pole 110 of VWC sensor 100 has a diameter 157 ranging between 10-40 mm (e.g., as shown in FIG. 5E). Helical blades 122 can have a diameter of 158 ranging between 80-120 mm. For example, diameter 158a of helical blade 122a can be greater than diameter 158b of helical blade 122b (e.g., as shown in FIG. 5E) such that helical blade 122a, which can follow a screwing path of helical blade 122b during a screwing motion of an installation process, enters a undisturbed soil, thereby providing a good contact between helical blade 122a and/or RF electrodes 124-2a and target soil 80.

In some embodiments, RF electrodes 124-2 (e.g., as shown in FIG. 5A) and/or RF electrodes 124 (e.g., as shown in FIGS. 1B-1C, FIG. 2A-2E, FIG. 4A-4D) positioned at predefined radial distance 154 from pole 110, as described above and schematically shown in FIG. 5E. Radial distance 154 can range between 18-40 mm and/or such that RF electrodes 124-2 and/or RF electrodes 124 being positioned at 30% most lateral portion of helical blades 122 (e.g., RF electrodes 124-2a, 124-2b secured to helical blades 122a, 122b as shown in FIG. 5E). Radial distance 154 (e.g., radial distance of RF electrodes 124, 124-2 from pole 110) and/or a radial distance 154a between the RF electrodes (e.g., RF electrodes 124-2a embedded within helical blade 122a, as shown in FIG. 5E) can be defined based on a desired RF field to be generated to measure a VWC of a target soil 80.

In some embodiments, helical blades 122a, 122b of VWC probes 120a, 120b are secured to pole 110 and separated by longitudinal distance 152 (e.g., as shown in FIG. 5E). Longitudinal distance 152 can be predefined to, for example, optimize the accuracy of VWC measurement of a target soil 80 and/or to provide a good contact between helical blades 122a, 122b and the target soil during a screwing motion of an installation process. For example, longitudinal distance 152 can be predefined such that helical blades 122a, 122b follow a same helical path along pole 110 as would if helical blades 122a, 122b being parts of a single helical blade (e.g., helical blade 110, as shown in FIG. 6). In some embodiments, longitudinal distance 152 has a value of k pitches 159, where k is an integer (e.g., as shown in FIG. 6). In some embodiments, k is greater or equal to 2 (k>2). Separation of helical blades 122a, 122b by longitudinal distance 152 can prevent continuous water flow along a whole length of pole 110 and provide at least two zones of target soil 80 (e.g., schematically separated by broken line 92 in FIG. 5E) through which water flow is discontinuous. In some embodiments, longitudinal distance 152 deviates by 2-4% from the value of k pitches 159 such that helical blade 122a) such that helical blade 122a, which can follow a screwing path of helical blade 122b during the screwing motion of the installation process, enters a undisturbed soil, thereby providing a good contact between helical blade 122a and/or RF electrodes 124a and a target soil, which does not follow a same screwing path of helical blade 122b during a screwing motion of an installation process, enters a undisturbed soil, thereby improving a contact between helical blade 122a and/or RF electrodes (e.g., RF electrodes 124-2a as shown in FIG. 5E) and target soil 80. These considerations may be applicable to any of the configurations of VWC sensors 100, including configurations with a central shaft (e.g., with pole 110). FIG. 6 further illustrates schematically that the distance between blades, indicated by 159a may correspond exactly or approximately to an integer number of pitches, represented schematically by the broken-line windings.

FIG. 6 is an illustration of configuration of volumetric water content (VWC) sensors 100a, 100b with a support 110 being a coreless helical blade, according to some embodiments of the invention. In some embodiments, coreless helical blade 110 has a tapered shape (e.g., as shown in FIG. 6). VWC sensors 100a, 100b can include at least one VWC probe 120, e.g., VWC probes 120a, 120b, as shown in FIG. 6. In some embodiments, VWC probes 120a, 120b are positioned concentrically along a longitudinal axis 155 of coreless helical blade 110 at predefined longitudinal locations and/or include RF electrodes 124a, 124b. In some embodiments, VWC probes 120a, 120b are VWC probes described in FIGS. 1-5. VWC sensors 100a, 100b can include at least one electronics unit (e.g., electronics unit 160 as shown in FIGS. 8-9). In some embodiments, the electronics units are embedded within coreless helical blade 110 of VWC sensors 100a, 100b.

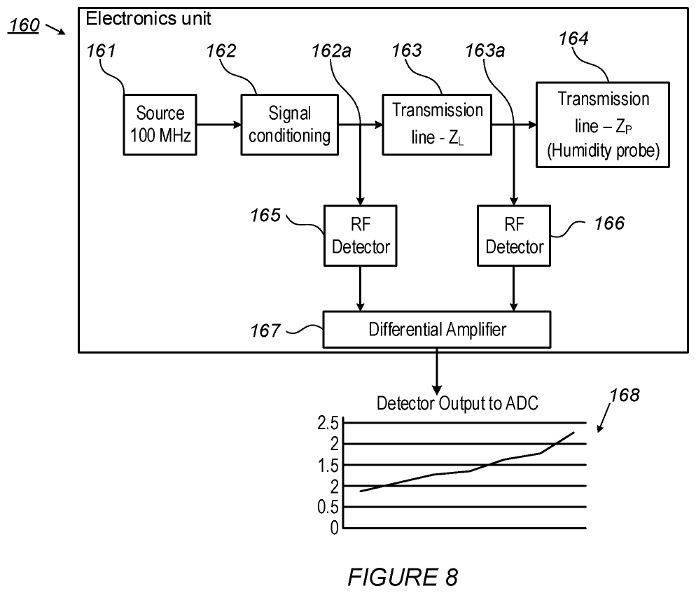

FIG. 7 is an illustration of a volumetric water content (VWC) probe 120 including segmented RF electrodes 124-3, according to some embodiments of the invention. FIG. 7 presents a top view of VWC probe 120. In some embodiments, RF electrodes of VWC probe 120 (e.g., RF electrodes 124, 124-1, and/or 124-2 as shown in FIGS. 1-6) are segmented RF electrodes (e.g., RF electrodes 124-3, as shown in FIG. 7) that are secured and/or embedded within helical blade 122. In some embodiments, VWC probe 120 has eight pairs of segmented RF electrodes 124-3 (e.g., pairs 124-3a . . . 124-3g as shown in FIG. 7). In some embodiments, an RF field can be generated and/or measured by segmented RF electrodes 124-3 of each pair. RF fields measured by each pair of RF electrodes 124-3a . . . 124-3g can be averaged to determine a VWC of a target soil. In some embodiments, RF field measured by at least one pair of segmented RF electrodes, for example, by pair 123-3c, can significantly differs from RF fields measured by the rest of the pairs, for example due to accommodation of air bubbles on RF electrodes of pair 123-3c. Accordingly, RF field measured by pair 123-3c can be excluded from averaging, thereby eliminating introduction of measurement errors.

One advantage of the present invention can include enabling a self-tapping installation of VWC sensor 100. The self-tapping installation can include pushing tapered tip 112 of VWC sensor 100 into a target soil and/or establishing a rotational motion of the sensor about its longitudinal axis. The rotational motion of helical blades 122 secured along VWC sensor 100 (e.g., as shown in FIG. 1) can generate a screwing action that can wind the sensor into the target soil, such that no pre-drilling procedures are required, which minimizes the disturbance of the soil thereof and reduces vertical flow of water along pole 110 and/or helical blades 120.

During an installation of VWC sensor 100, a target soil can be disturbed in a vicinity of pole 110. Disclosed VWC sensor 100 can include RF electrodes 124 secured to helical blades 122 of VWC probes 120 at predefined radial distances from pole 110 (e.g., as shown in FIG. 1). Accordingly, another advantage of the present invention is that it can enable alienating the VWC measurement (e.g., by RF electrodes 124) away from pole 110 such that measurements of undisturbed soil are performed.

During an installation of VWC sensor 100, a target soil can also be disturbed in a vicinity of helical blades 122. Disclosed VWC sensor 100 can include RF electrode 124 secured to an outer lateral side of helical blade 122 and at least one RF electrode 124-2 embedded within the same helical blade 122 such that embedded RF electrodes 124-2 protrude above at least one of surfaces of the blade (e.g., as shown in FIGS. 5A-5B). Accordingly, another advantage of the present invention is that it can enable alienating the VWC measurement (e.g., by lateral RF electrodes 124 and protruding RF electrodes 124-2) away from the surfaces of helical blades 122 such that it can allow measurement of undisturbed soil.

FIG. 8 is a schematic block diagram illustrating an electronics unit 160 of volumetric water content (VWC) sensor 100, according to some embodiments of the invention. Electronics unit 160 illustrated in FIGS. 8-9 is an enablement to a transmission line based on ADR electronic circuit as described below. Alternatively, electronics unit 160 can be an enablement to a transmission line based on amplitude domain reflectometry (ADR), time domain reflectometry (TDR), frequency domain reflectometry (FDR) and/or time domain transmission (TDT) electronic circuits. In some embodiments, electronics unit 160 can be an enablement to a capacitance probe.

An RF signal can be generated by a source 161 (e.g., an oscillator). In some embodiments, the generated RF signal has a frequency of 100 MHz. The generated RF signal can be transmitted to a signal conditioning unit 162 (e.g., a filter) to create a filtered RF signal. The filtered RF signal can be transmitted through a first transmission line 163 (e.g., phase shifter) and/or through a second transmission line 164 to a target soil. In some embodiments, at least a portion of transmission line 164 is at least one VWC probe 120 (e.g., as disclosed in FIG. 1, FIGS. 2A-2E, FIGS. 4A-4C and/or FIG. 5). In some embodiments, first transmission line 163 has an impedance value of Z_{L} and/or second transmission line 164 has an impedance value of Z_{P}.

The impedance Z_{P} of transmission line 164 can be based on a relative dielectric constant ε of the target soil that surrounds transmission line 164. The relative dielectric constant ε can be based on a VWC level of the target soil. For example, Equation 1 shows the impedance Z_{P} of the transmission 164 line as follows:

$${Z}_{P} ∝ \frac{1}{\sqrt{ɛ}} \qquad ( Equation 1 )$$

A reflection coefficient ρ of transmission line 163 and transmission line 164 can be based on Z_{L}, Z_{P}. For example, Equation 2 shows the reflection coefficient ρ as follows:

$$ρ = \frac{{Z}_{P} - {Z}_{L}}{{Z}_{P} + {Z}_{L}} \qquad ( Equation 2 )$$

A voltage value Vₒ (e.g., the filtered RF signal) at a junction 162a of filter 162 and transmission line 163 and/or a voltage value V_{P} at a junction 163a of transmission line 163 and transmission line 164 can be based the reflection coefficient ρ. For example, Equation 3 and Equation 4 show the voltage value V₀ and the voltage value V_{P} as follows:

Vₒ∝(1−ρ) (Equation 3)

V_{P}∝(1+ρ) (Equation 4)

The voltage value Vₒ can also be based on forward voltage value V_{FWD} and reflected voltage value V_{REF}. For example, Equation 5 shows the voltage value V₀ as follows:

Vₒ=V_{FWD}+V_{REF} (Equation 5)

The voltage value Vₒ and/or the voltage value V_{P} can be measured by respective RF detectors 165, 166 and transmitted to a differential amplifier 167 to generate a differential voltage value ΔV=Vₒ−V_{P}. The differential voltage value ΔV can be based on the reflective coefficient ρ and as a result can be based on the dielectric constant ε of the VWC level of the target soil, such that allowing determining the value of e. For example, Equation 6 shows the differential voltage value ΔV as follows:

ΔV=Vₒ−V_{P}∝2ρ∝ϵ (Equation 6)

An example of a dependence of differential voltage value ΔV on the VWC level ε of the target soil is illustrated in graph 168.

FIG. 9 is a schematic block diagram of an electronic circuitry of electronics unit of volumetric water content (VWC) sensor 100, according to some embodiments of the invention. An oscillator 161 can generate a RF signal. The generated RF signal can be filtered by a filter 162 to generate a filtered RF signal. The filtered RF signal can be transmitted through a phase shifter 163 (e.g., that can act as a transmission line) and through a second transmission line 164 to a target soil.

Second transmission line 164 can include a switch 164-1 and/or a controller 164-2. Controller 164-2 can control switch 164-1 to connect phase shifter 163 to at least one of: a phase shifter 164-3a, a phase shifter 164-3b, a first reference load 164-4a and/or a second reference load 164-4b. In some embodiments, phase shifter 164-3a is connected to VWC probe 120a and/or phase shifter 164-3b is connected to VWC probe 120b, where VWC probes 120a, 120b can be VWC probes 120 disclosed in FIG. 1, FIGS. 2A-2E, FIGS. 4A-4C and/or FIG. 5. In some embodiments, VWC probes 120a, 120b are positioned at opposing ends along a longitudinal axis of VWC sensor 100,100a.

A voltage value Vₒ of the filtered RF signal can be measured by a peak detector 165 at a junction 162a of filter 162 and phase shifter 163 and/or a voltage value V_{P} at a junction 163a of phase shifter 163 and transmission line 164 can be measured by a peak detector 166. The voltages values Vₒ and V_{P} can be transmitted to a differential amplifier 167 to generate a differential voltage value ΔV. The voltage value V_{P}, and as a result differential voltage value ΔV can be a function of the level of VWC ε of the target soil, as disclosed above (e.g., in Equations 1-6).

In some embodiments, phase shifters 164-3a, 163-3b rectify phase shifts that can be caused by a physical distance between junction 163a (where voltage value V_{P} is measured) and VWC probes 120a, 120b. In some embodiments, reference loads 164-4a, 164-4b are used for a calibration of soil sensor 100.

In some embodiments, the differential voltage value ΔV is digitalized by an analog to digital converter (ADC) 169 and/or transmitted to an external system 90 (e.g., cloud network).

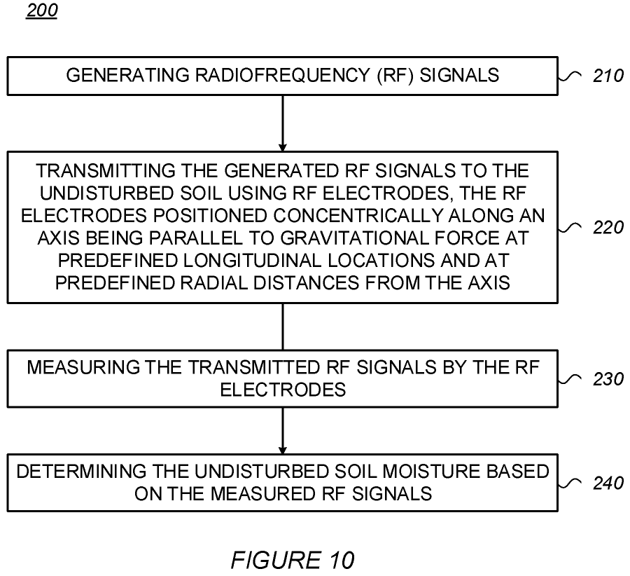

FIG. 10 is a flowchart illustrating a method 200 of measuring a undisturbed volumetric water content (VWC), according to some embodiments of the invention. In some embodiments, method 200 can be carried out using VWC sensor 100 described above (e.g., as shown in FIGS. 1-7).

Method 200 can include generating 210 radiofrequency (RF) signals. Method 200 can include transmitting 220 the generated RF signals to the undisturbed soil using RF electrodes, the RF electrodes positioned concentrically along an axis being parallel to gravitational force at predefined longitudinal locations and at predefined radial distances from the axis.

In some embodiments, the RF electrodes have a helical shape. In some embodiments, the RF electrodes secured to helical blades, where the helical blades can be positioned concentrically along the axis at the predefined longitudinal locations. In some embodiments, the at least one of the RF electrodes is secured to an outer lateral side of the at least one of the helical blades. In some embodiments, the at least one of the RF electrodes is embedded within the at least one of the helical blades such that the at least one of the embedded RF electrodes protrudes above at least one of surfaces of that helical blade.

Method 200 can include measuring 230 the transmitted RF signals by the RF electrodes. Method 200 can include determining 240 the undisturbed VWC based on the measured RF signals.

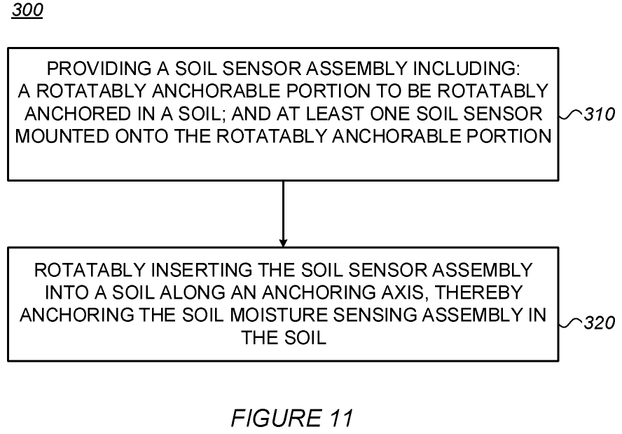

FIG. 11 is a flowchart illustrating a method 300 of installing a soil sensor assembly, according to some embodiments of the invention. Method 300 can include providing 310 a soil sensor assembly including: a rotatably anchorable portion to be rotatably anchored in a soil; and at least one soil sensor mounted onto the rotatably anchorable portion. Method 300 can include rotatably inserting 320 the soil sensor assembly into a soil along an anchoring axis, thereby anchoring the soil sensing assembly in the soil.

In some embodiments, the rotatably anchorable portion includes at least one threading arranged about the anchoring axis, the at least one threading includes at least one blade portion extending outwardly from the anchoring axis, wherein at least one soil sensor is located on the at least one of the blade portions, and wherein the rotatably inserting of the soil sensor assembly into the soil along the anchoring axis, thereby anchoring the soil sensor assembly in the soil, is operative to bring the at least one soil sensor located on the at least one of the blade portions into a soil sensing engagement with a portion of the soil which is substantially undisturbed.

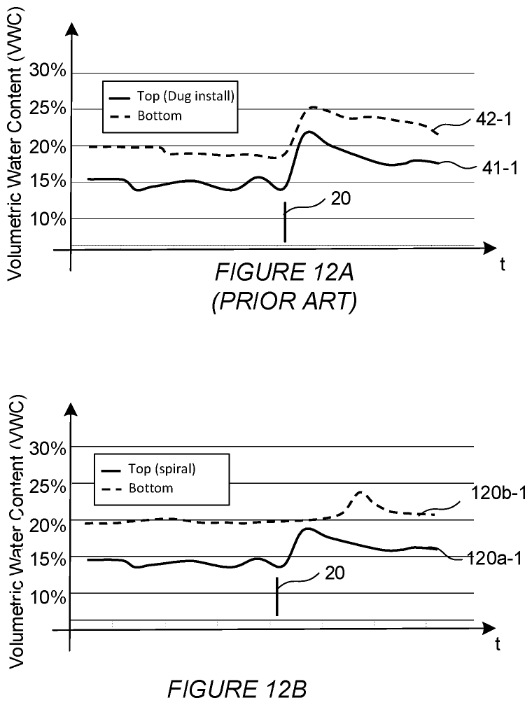

FIG. 12A is a graph illustrating volumetric water content (VWC) measurement results being measured by a prior art profile sensor 40, according to the prior art. FIG. 12B is a graph illustrating volumetric water content (VWC) measurement results being measured by a VWC sensor 100, according to some embodiments of the invention.

Typically, following an irrigation event 20, measurements from bottom sensor 42 of prior art profile sensor 40 (e.g., as shown in FIG. 1A) can erroneously show a rise in VWC of a disturbed target soil (e.g., line 42-1 as shown in FIG. 12A) that can be similar in timing and amplitude, to measurements of top sensor 41 (e.g., line 41-1 as shown in FIG. 12A). Such measurements can be biased, since water takes time to filtrate down through undisturbed soil.

In contrast, the disclosed sensors were found to be sensitive and indicate irrigation events. Following an irrigation event 20, the VWC measurements generated by the VWC sensor 100 clearly show delay in timing between measurement of top sensor 120a (e.g., line 120a-1 as shown in FIG. 12B) and measurement of bottom sensor 120b (e.g., line 120b-1 as shown in FIG. 12B), which emphasizes that a target soil is undisturbed during an installation of VWC sensor 100.

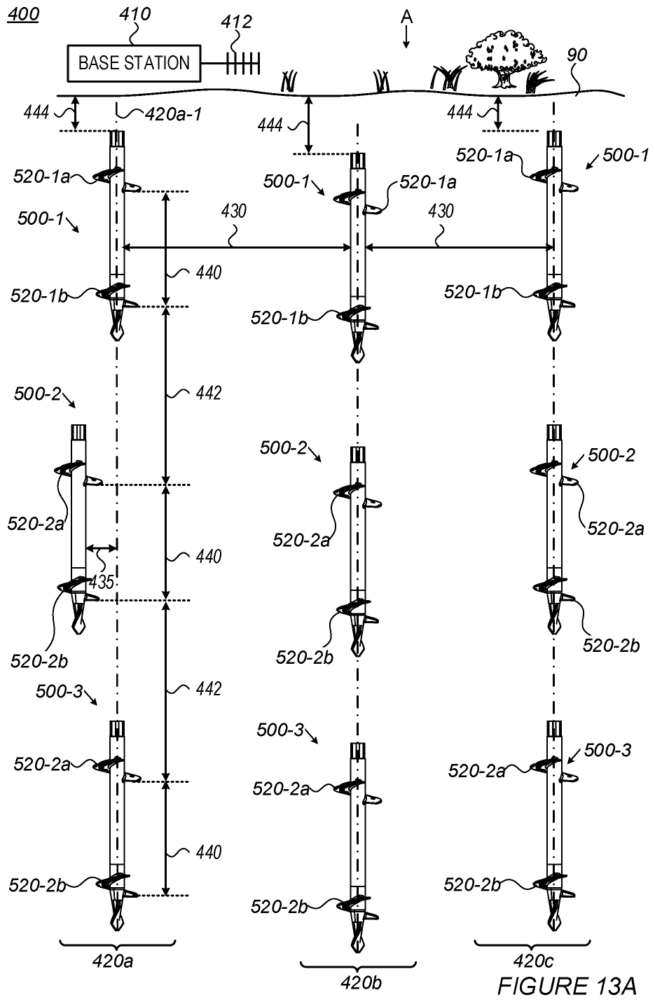

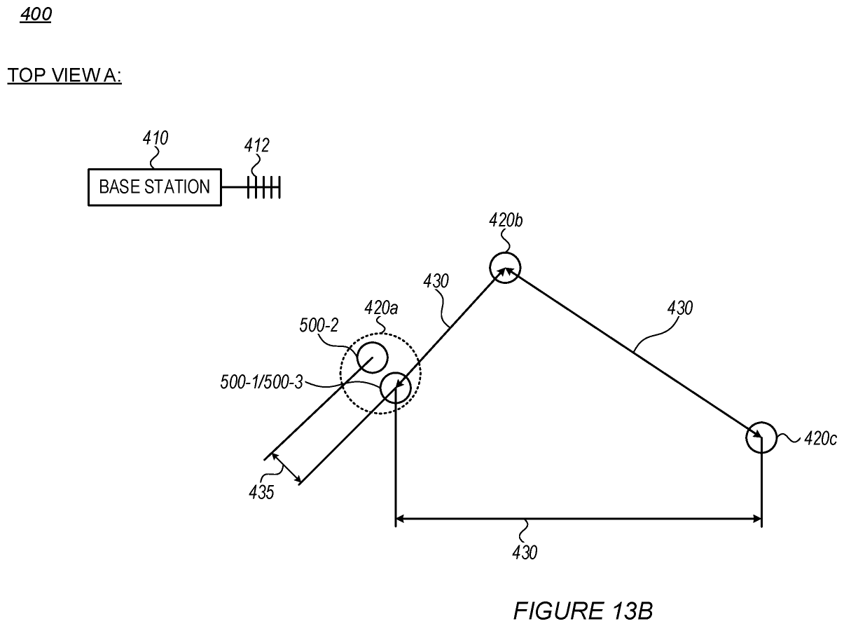

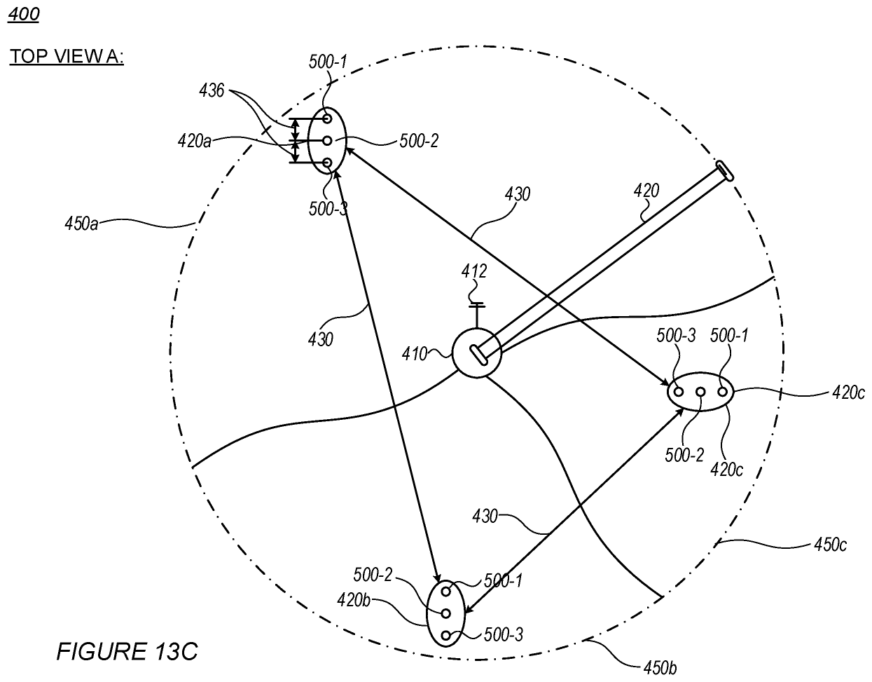

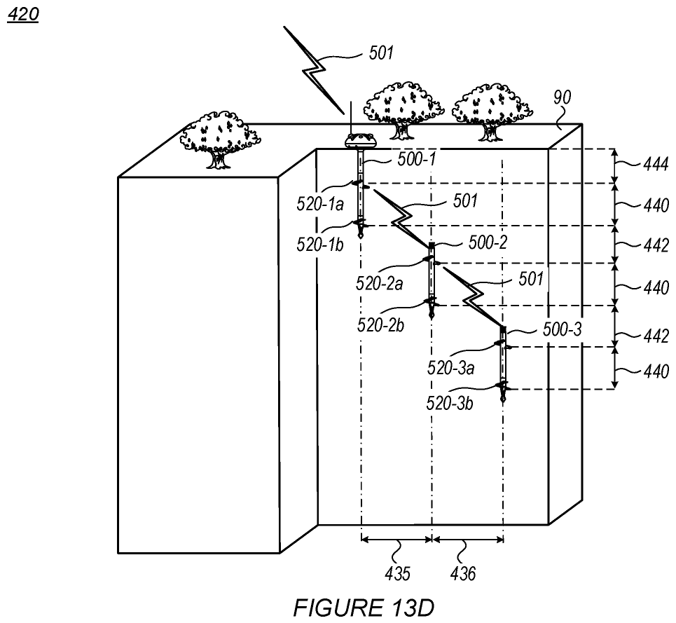

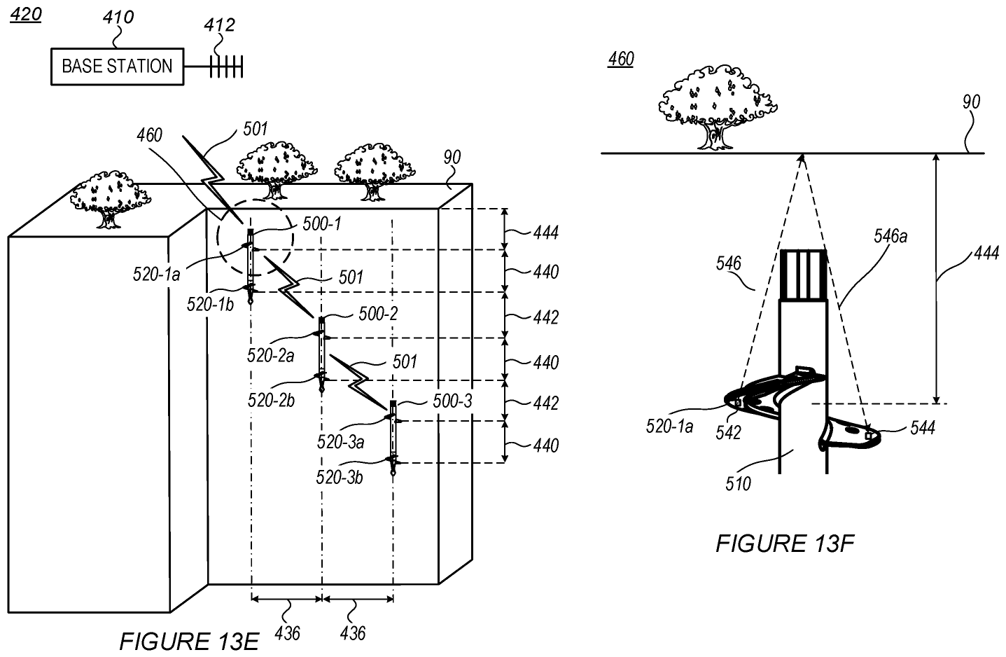

FIGS. 13A-13C are illustrations of an underground soil sensors system 400, according to some embodiments of the invention. FIG. 13A provides a side view and FIGS. 13B-13C provide a top view of underground soil sensors system 400, respectively.

Underground soil sensors system 400 can include a base station 410. Base station 410 can include at least one antenna 412 that can receive and/or transmit signals. In some embodiments, the signals are a radiofrequency (RF) signals.

Underground soil sensors system 400 can include at least one set 420 of soil sensors, for example, sets 420a, 420b, 420c as shown in FIGS. 13A-13B. Soil sensors in sets 420a, 420b, 420c can be positioned at a predetermined vertical distance below a surface 90 of a target soil. FIGS. 13A-13C illustrate three sets of soil sensors (e.g., sets 420a, 420b, 420c), where each of the sets includes three soil sensors (e.g., soil sensors 500-1, 500-2, 500-3), however this in not meant to be limiting in any way and underground soil sensors system 400 can include any number of sets, where each of the sets can include any number of soil sensors, and where each of the soil sensor can include any sensor type as described below.

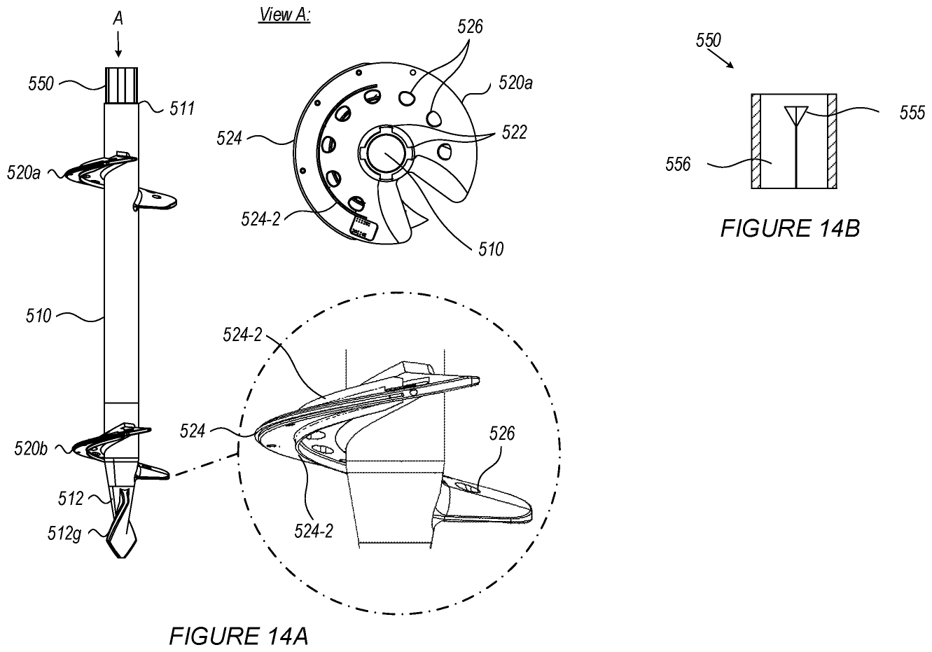

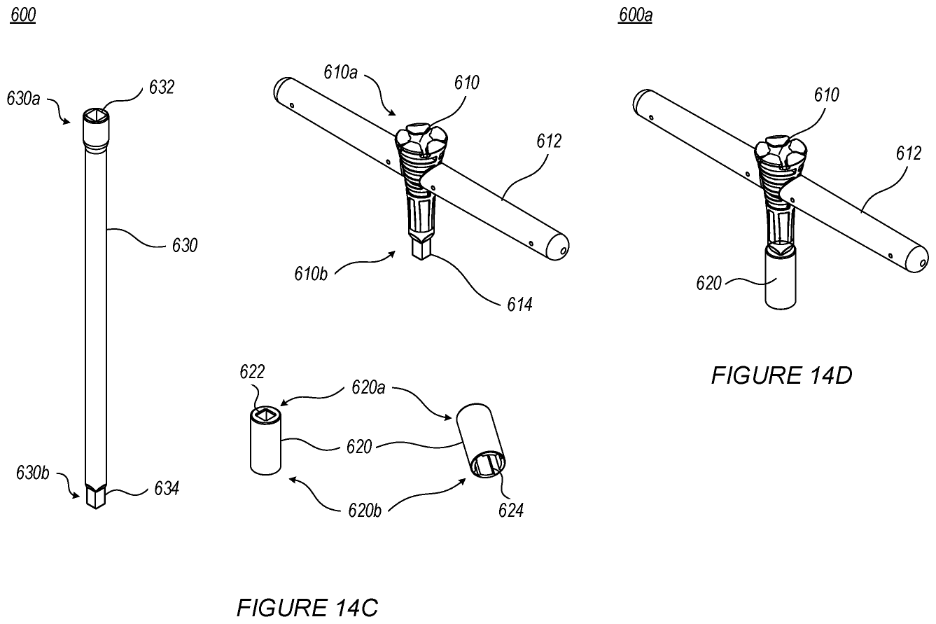

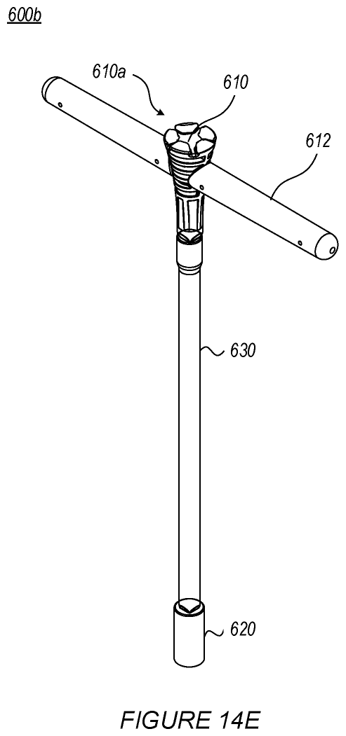

FIG. 14A is an illustration of a soil sensor 500, according to some embodiments of the invention. Soil sensors 500 can be part of underground soil sensors system 400, for example as shown in FIG. 13A. FIG. 14B is an illustration of a cross-section of an installing tool interface 550 of a soil sensor 500, according to some embodiments of the invention.

Soil sensor 500 can include at least portions of VWC sensor 100 as described in detail with respect to FIGS. 1A-1C, FIGS. 2A-2E, FIGS. 3A-3B, FIGS. 4A-4D, FIGS. 5A-5E and/or FIGS. 6-7. For example, soil sensor 500 can include a support 510 that can be rotatably anchored in target soil. Soil sensor 500 can include soil probes 520. Each of soil probes 520 can include helical blade 522, at least one RF electrode 524 secured to an outer lateral side of helical blade 522 and/or at least one RF electrode 524-2 embedded within helical blade 122 at a predefined radial distance from support 510 such that embedded RF electrodes 524-2 protrude above at least one of surfaces of helical blade 122. Helical blades 522 can include a plurality of holes 526 positioned between support 510 and protruding RF electrodes 524-2 and/or RF electrodes 524 to drain water flowing along support 510 and/or along helical blades 522. In some embodiments, soil sensor 500 and/or soil probes 520 include a volumetric water content (VWC) sensor, a temperature sensor, a pH sensor, a pressure sensor, a salinity sensor, a sensor for determining level of minerals in a target soil and/or any combination thereof. Tip 512 of soil sensor 500 can include a helical blade 512g.

Soil sensor 500 can include an installing tool interface 550 positioned at a first end 511 of support 510 (e.g., as shown in FIG. 14A). Installing tool interface 550 can include connector 552 to enable a connection of an installing tool to support 510 of soil sensor 500 (e.g., as described in detail with respect to FIGS. 14C-14E). Connector 552 can include any connection means known in the art. In some embodiments, connector 552 includes protrusions (e.g., as shown in FIG. 14A).

Installing tool interface 550 can include at least one antenna 555 to transmit signals to antenna 412 of base station 410 (e.g., as shown in FIG. 14B). Installing tool interface 550 can also include an air gap 556 surrounding antenna 555 to improve a quality of transmitted signals (e.g., as shown in FIG. 14B).

FIG. 14C-14E are illustrations of an installing tool 600 for a soil sensor 500, according to some embodiments of the invention. FIG. 14C presents an isometric view of installing tool 600.

Installing tool 600 can include a first section 610 having a first end 610a and a second end 610b. First section 610 can include a handle 612 detachably connectable to the first section at first end 610a. First section 610 can also include a connector 614 at second end 610b. In some embodiments, handle 612 is used to establish a rotational motion of installing tool 600 and/or soil sensor 500 during an installation of the sensor.

Installing tool 600 can include a second section 620 having a first end 620a and a second end 620b. Second section 620 can include a connector 622 at first end 620a and/or a connector 624 at second end 620b. In some embodiments, connector 614 of first section 610 matches connector 622 of second section 620 such that first section 610 can be detachably connected to second section 620 to provide installing tool 600a (e.g., as shown in FIG. 14D). In some embodiments, connector 624 of second section 620 matches connector 552 of installing tool interface 550 of soil sensor 500 such that installing tool 600a can be detachably connected to the soil sensor.

Installing tool 600 can include a third section 630 having a first end 630a and a second end 630b. Third section 630 can include a connector 632 at first end 630a and/or a connector 634 at second end 630b. Connector 632 of third section 630 can match connector 614 of first section such that first section 610 can be detachably connected to third section 630. Connector 634 of third section can match connector 622 of second section 620 such that third section 630 can be detachably connected to second section 620. Connection of first section 610 to third section 630 and/or connection of third section 630 to second section 620 can provide installing tool 600b, as shown in FIG. 14E.

In some embodiments, installing tool 600b has a substantially greater length as compared with installing tool 600a. Accordingly, installing tool 600b can be used to install soil sensor 500 deeper in the target soil as compared to installing tool 600a. In some embodiments, two or more third sections 630 can be detachably interconnected (e.g., using connectors 632, 634) to increase a length of installing tool 600b.

Reference is now made back to FIGS. 13A-13C. In some embodiments, soil sensor 500-1 in sets 420a, 420b, 420c is a topmost sensor (e.g., soil sensor that is positioned closer to surface 90 of the target soil) and sensor 500-3 is a bottommost sensor (e.g., sensor that is positioned deepest below surface 90 of the target soil). Soil sensors 500-1, 500-2, 500-3 can transmit and/or receive signals. In some embodiments, the signals include electromagnetic (EM) signals, radiofrequency (RF) signals, ultrasonic (US) signals, infrared (IR) signals and/or near infrared (NIR) signals. Topmost soil sensor 500-1 in each of sets 420a, 420b, 420c can also transmit signals to antenna 412 of base station 410.

Soil sensors 500-1, 500-2, 500-3 in sets 420a, 420b, 420c can be substantially aligned along a vertical axis of that set, for example, along vertical axes 420a-1, 420b-1, 420c-1, respectively, as shown in FIG. 13A. Vertical axes 420a-1, 420b-1, 420c-1 can be substantially parallel to gravitational force. In some embodiments, all the soil sensors in the at least one of the sets are aligned along the vertical axis of that set. For example, soil sensors 500-1, 500-2, 500-3 in sets 420b, 420c can be aligned along vertical axes 420b-1, 420c-1, respectively, as shown in FIG. 13A. In some embodiments, at least one soil senor in the at least one of the sets can have an offset in a horizontal direction from the vertical axis of that set, where the horizontal direction is perpendicular to gravitational force. For example, soil sensor 500-2 in set 420a can be positioned at a horizontal offset distance 435 from vertical axis 420a-1 (e.g., as shown in FIGS. 13A-13B).

In some embodiments, soil sensors 500-1, 500-2, 500-3 in at least one of sets 420a, 420b, 420c are positioned at predetermined horizontal distance 436 from each other, for example as shown in FIG. 13C. Each of sets 420a, 420b, 420c can be positioned in a different irrigation zone 450a, 450b, 450c in a field. In some embodiments, base station 410 of underground soil sensors system 400 is positioned on a pivot 420 that irrigates irrigation zones 430a, 430b, 430c.

Each of sets 420a, 420b, 420c can be positioned at a predetermined horizontal distance 430 from an adjacent set and/or adjacent sets (e.g., distance 430 between adjacent sets 420a, 420b, adjacent sets 420a, 420c and/or adjacent sets 420b, 420c; e.g., as shown in FIG. 13B-13C). In some embodiments, horizontal distance 430 is predetermined, for example, to avoid interference between transmissions of signals in the adjacent sets (e.g., interference between soil sensor 500-3 in set 420a and/or soil sensor 500-2 is set 420c). In some embodiments, horizontal offset distance 435 and/or horizontal distance 436 is less than 10% of horizontal distance 430 between the adjacent sets (e.g., as shown in FIGS. 13B-13C). In some embodiments, horizontal distance 430 between two adjacent sets is greater and/or smaller than horizontal distance 430 between two other adjacent sets. For example, horizontal distance 430 between adjacent sets 420a, 420b can be smaller than horizontal distance 430 between adjacent sets 420b, 420c (e.g., as shown in FIG. 13B).

Each soil sensor in each of the sets can transmit signals to an adjacent soil sensor positioned thereabove in that set and/or to receive signals from an adjacent soil sensor positioned therebelow in that set. For example, soil sensor 500-3 in set 420a can transmit signals to soil sensor 500-2 in set 420a, and soil sensor 500-2 can receive signals from sensor 500-3 and/or transmit signals to soil sensor 500-1. In another example, soil sensor 500-1 can receive signals from sensor 500-2 and/or transmit signals to base station 410.

The signals being transmitted by each of the soil sensors in each of the sets can include information regarding at least one of: a volumetric water content (VWC), a temperature, a pH, a pressure, a salinity, a level of minerals of the target soil and/or any combination thereof. The signals being transmitted by each of the soil sensors in each of the sets to the adjacent soil sensor positioned thereabove in that set can include information received from that soil sensor and/or from all soil sensors positioned therebelow in that set. For example, soil sensor 500-2 in set 420a can transmit signals to soil sensor 500-1 in set 420a, where the signals can include information received from soil sensor 500-2 and/or from soil sensor 500-3 positioned below soil sensor 500-2 in set 420a. In another example, soil sensor 500-1 in set 420a can transmit signals to base station 410, where the signals can include information received by soil sensor 500-1, and/or soil sensors 500-2, 500-3 positioned below soil sensor 500-1 in set 420a.

The signals being transmitted by each of the soil sensors 500-1, 500-2, 500-3 in each of the sets 420a, 420b, 420c can include an identifying information. The identifying information of each of the soil sensors can include, for example, an identification code. In some embodiments, the identification code of each of the soil sensors 500-1, 500-2, 500-3 in each of the sets 420a, 420b, 420c is related to a location information of that soil sensor (e.g., a horizontal and/or vertical position with respect, for example, to base station 410), where the location information can be stored in base station 410.

In some embodiments, each of the soil sensors 500-1, 500-2, 500-3 in each of the sets 420a, 420b, 420c transmits signals at different time sequences, different frequencies, with different spreading codes and/or any combination thereof to avoid an interference between the signals transmitted by the soil sensors in that set (e.g., the interference between soil sensors 500-1, 500-2, 500-3 in set 520a) and/or between the soil sensors in the adjacent sets (e.g., the interference between soil sensor 500-2 in set 420b and sensor 500-1 in set 420c).

Soil sensors 500-1, 500-2, 500-3 in sets 420a, 420b, 420c can include at least two soil probes separated by a vertical distance 440 along that soil sensor, for example, soil probes 520-1a, 520-1b in soil sensor 500-1 in set 420a, as shown in FIG. 13A. In some embodiments, each of the soil probes of each of the soil sensors in the at least one set can transmit signals to an adjacent soil probe positioned thereabove in that soil sensor and can receive signals from an adjacent soil probe positioned therebelow in that soil sensor. For example, probe 520-1b of soil sensor 500-1 (e.g., in set 420a) can transmit signals to probe 520-1a of soil sensor 500-1 and/or probe 520-1a of soil sensor 500-1 can receive signals from probe 520-1b of soil sensor 500-1.

In some embodiments, a topmost soil probe of each of the soil sensors in the at least one set can transmit signals to a bottommost soil probe of the adjacent soil sensor positioned thereabove in that set and wherein a bottommost soil probe of that soil sensor to receive signals from a topmost soil probe of the adjacent soil sensor positioned therebelow in that set. For example, soil probe 520-2a of soil sensor 500-2 (e.g., in set 420a) can transmit signals to soil probe 520-1b of soil sensor 500-1 and/or soil probe 520-3a of soil sensor 500-3 can transmit signals to soil probe 520-2b of soil sensor 500-2.

In some embodiments, the soil sensors can be positioned within the target soil such that there is a vertical distance 442 between a bottommost soil probe of each of the soil sensors in the at least one set and a topmost soil probe of the adjacent soil sensor positioned therebelow in that set (e.g., vertical distance 442 between soil probe 520-1b of soil probe 500-1 (e.g., in set 420a) and soil probe 520-2b of soil probe 500-2; e.g., as shown in FIG. 13A and FIGS. 13D-13E). In some embodiments, distance value 442 is equal to distance value 440 (e.g., as shown in FIGS. 13D-13E). In some embodiments, topmost soil sensor 500-1 in each of sets 420a, 420b, 420c can be positioned at a vertical distance 444 below surface 90 of the target soil (e.g., as shown in FIG. 13A). In some embodiments, topmost soil sensor 500-1 of at least one of sets 420a, 420b, 420c is positioned deeper below surface 90 of the target soil than in other sets. For example, distance 444 of topmost sensor 500-1 in set 420b can be greater than distance 444 of topmost senor 500-1 is sets 420a, 420c (e.g., as shown in FIG. 13A). In some embodiments, soil sensors 500-1, 500-2, 500-3 in each of sets 420a, 420b, 420c are positioned below surface 90 of the target soil (e.g., as shown in FIG. 13A). In some embodiments, at least a portion of at least one of the soil sensors in at least one of the sets is positioned above surface 90 of the target soil (e.g., as shown in FIG. 13E below).

FIGS. 13D-13E are illustrations of a set 420 of soil sensors 500, according to some embodiments of the invention. Set 420 can be a part of underground soils sensors system 400. For example, set 420 can be any of sets 420a, 420b, 420c as shown in FIGS. 13A-13C. Set 420 can include soil sensors 500-1, 500-2, 500-3 and/or any number of sensors 500 positioned at predetermined vertical distance below surface 90 of the target soil.

In some embodiments, vertical distance 442 between a bottommost soil probe of each of the soil sensors and topmost soil probe of the adjacent soil sensor positioned therebelow in that set (e.g., vertical distance 442 between soil probe 520-1b of soil probe and soil probe 520-2b of soil probe 500-2) is equal to vertical distance 440 between the soil probes of each of the soil sensors (e.g., vertical distance 440 between soil probes 520-1a, 520-1b of soil sensor 500-1). In some embodiments, distance 444 between topmost soil probe 520-1a of topmost soil sensor 500-1 and surface 90 of the target soil has the same value as vertical distance 440 and/or vertical distance 442, for example as shown in FIG. 13E. In some embodiments, vertical distance 440 and/or vertical distance 442 range between 90-350 mm. In some embodiments, soil sensors 500-1, 500-2, 500-3 in set 420 are positioned at predetermined horizontal distance 436 from each other (e.g., as shown in FIGS. 13C-13D).

In some embodiments, topmost soil sensor 500-1 includes an electronics bay 530 (e.g., electronics bay 130 as described in detail with respect to FIGS. 1B-1C). Topmost soil sensor 500-1 can be installed such that electronic bay 530 is positioned above surface 90 of the target soil (e.g., as shown in FIG. 13D).

FIG. 13F is an illustration of an inverse ground-penetrating radar (IGPR) tool 540 in a topmost sensor 500-1 in set 420 of underground soil sensors system 400, according to some embodiments of the invention. FIG. 13F presents an enlarged region 460 represented by a dashed circle in FIG. 13E.

In some embodiments, topmost soil sensor 500-1 in set 420 is positioned below surface 90 of the target soil (e.g., as shown in FIGS. 13A, 13E) at predetermined distance 444. In some embodiments, distance 444 ranges between 10-60 cm. Topmost soil sensor 500-1 can include an inverse ground-penetrating radar (IGPR) tool 540 to measure desired soil properties (e.g., a VWC) of a soil between soil sensor 500-1 and surface 90. IGPR tool 540 can be coupled, for example, to topmost soil probe 520-1a of topmost soil sensor 500-1. IGPR tool 540 can include a transmitting element 542 and/or receiving element 544 to transmit and receive electromagnetic (EM) signal, respectively. In some embodiments, each of transmitting and/or receiving elements 542, 544 can transmit and/or receive EM signals.

Transmitting element 542 can transmit an EM signal 546 that can at least partly reflect from surface 90 of the target soil due to, for example, impedance difference between the soil and an air. A reflected EM signal 546a can be received by the receiving element 544 of IGPR tool 540. IGPR tool 540 can determine, based on a time difference between transmission of EM signal 546 (e.g., by transmitting element 542) and detection of reflected EM signal 546a (e.g., by receiving element 544), the desired properties of the target soil (e.g., a VWC).

Reference is now made back to FIGS. 13E-13F. In some embodiments, each of the soil sensors in each of the sets (e.g., soil sensor 500-2 in set 420) compares received signal quality information from an adjacent soil sensor, positioned therebelow in that set (e.g., soil sensor 500-3 in set 420), with expected quality information. A change in signal quality (e.g., between the quality of received signal and the expected quality of the signal) can be an indicator of a measured soil property (e.g., a VWC) of an inter-sensor soil (e.g., the soil between soil sensors 500-2, 500-3 in set 420). Information regarding the change in the signal quality can be transmitted to an adjacent soil sensor positioned thereabove in that set (e.g., as described above) and/or transmitted by a topmost sensor in that set (e.g., soil sensor 500-1 in set 420) to base station 410. In some embodiments, the comparison of the change in signal quality is performed between signals received from an adjacent soil probes of each of the soil sensors (e.g., between probes 520-2a, 520-2b of soil sensor 500-2).

In some embodiments, the signal quality includes signal intensity. For example, signal transmitted by soil sensor 500-2 (e.g., signal indicated by arrow 501 in FIG. 13D) can include information regarding the signal intensity. Sensor 500-1 can receive signal 501, determine the intensity of the received signal, and/or compare the intensity of the transmitted signal and the received signal. A change in the intensity between transmitted signal 501 (e.g., transmitted by soil sensor 500-2) and received signal 501 (e.g., received by soil sensor 500-1) can be an indicator of the measured soil property (e.g., a VWC) of the soil between soil sensors 500-1, 500-2.

In some embodiments, transmitted signal 501 is attenuated and/or amplified while propagating through a target soil, depending on a type of the signal and/or on properties of the target soil. For example, a RF signal can be attenuated and ultrasonic (US) signal can be amplified while propagating in the target soil, depending for example, on a VWC of the soil. In some embodiments, soil sensors 500 can transmit and/or receive signals of various types, for example, RF and/or US signals.

In some embodiments, a measured soil property (e.g., a VWC) of an inter-sensor soil (e.g., the soil between soil sensors 500-2, 500-3 in set 420) is determined based on a quality of at least two signals, where each of the at least two signals is of different signal type. For example, soil sensors 500-3 can transmit a RF signal and a US signal to soil sensor 500-2 positioned thereabove in the set (e.g., set 420). In various embodiments, the RF signal is attenuated and the US signal is amplified while propagating in a target soil. Soil sensor 500-2 can receive the RF and US signals (e.g., transmitted by soil sensor 500-3), determine an intensity of the received RF and US signals and/or compare the determined intensities between the transmitted and received RF and US signals. Sensor 500-2 can also determine, based on the comparison of the intensities of the transmitted and received RF and US signals, the measured soil property of the inter-sensor soil. One advantage of determining the measured soil property of the inter-sensor soil based on the comparison of quality of two signals of different types (e.g., the RF and US signals) can include improving an accuracy of the soil measurements.

In some embodiments, the signal quality includes number of packets and/or the measured property of the soil (e.g., a VWC) is determined based on a change in packets number between transmitted signal 501 (e.g., transmitted by soil sensor 500-2) and received signal 501 (e.g., received by soil sensor 500-1).

In some embodiments, each of soil sensors 500-1, 500-2, 500-3 includes IGPR tool 540 (e.g., as described in detail with respect to FIG. 13F) to determine the measured soil property (e.g., a VWC) of the inter-sensor soil (e.g., the soil between soil sensors 500-2, 500-3 in set 420).

One advantage of the present invention can include providing an underground soil sensors system (e.g., underground soil sensors system 400) to perform profile measurements of desired soil properties (e.g., VWC of the soil). In some embodiments, all the soil sensors (e.g., soil sensors 500) in the underground soil system are positioned below the surface of the target soil (e.g., as shown in FIGS. 13A, 13E), thereby eliminating a need in uninstalling the system, for example during harvesting. In some embodiments, the disclosed underground soil sensors system is kept within a target soil for a period ranging between 8-15 years. In embodiments, where a portion of a topmost sensor in the underground soil sensors system is positioned above the surface of the soil (e.g., electronic bay 530 of sensor 500-1, as shown in FIG. 13D), only the topmost sensor can be uninstalled, for example during harvesting, and installed again thereafter. Another advantage of the present invention can include installing the disclosed soil sensors (e.g., soil sensors 500) without disturbing the target soil, thereby providing robust and/or accurate measurements of the undisturbed soil properties.

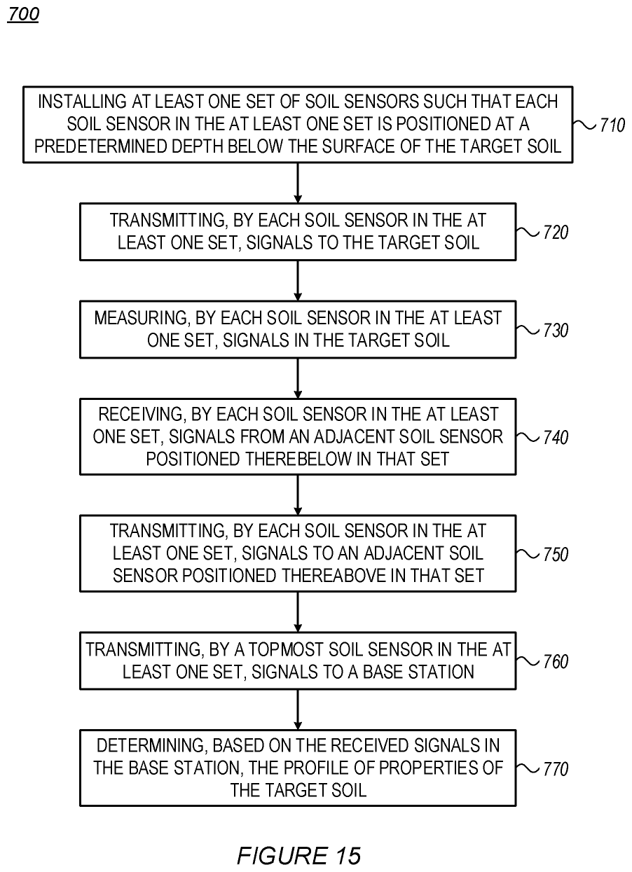

FIG. 15 is a flowchart illustrating a method 700 of determining a profile of properties of a target soil, according to some embodiments of the invention.

Method 700 can include installing 710 at least one set of soil sensors (e.g., sets 420a, 420b, 420c, as shown in FIG. 13A) such that each soil sensor (e.g., soil sensors 500-1, 500-2, 500-3) in the at least one set is positioned at a predetermined depth below the surface of the target soil.

In some embodiments, a longitudinal axis of each soil sensor in the at least one set is substantially aligned along a longitudinal axis of the topmost soil sensor in that set (e.g., vertical axes 420a-1, 420b-1, 420c-1, as shown in FIG. 13A), and wherein the longitudinal axis of the topmost soil sensor in the at least one set is substantially parallel to gravitational force (e.g., as shown in FIG. 13A). In some embodiments, a horizontal distance between two adjacent sets (e.g., horizontal distance 430, as shown in FIGS. 13A-13B) of soil sensors having a predetermined value. In some embodiments, the horizontal distance value (e.g., horizontal distance 430) is predetermined to avoid interference between transmissions of signals in the two adjacent sets. In some embodiments, a horizontal offset (e.g., horizontal distance 435, as shown in FIGS. 13A-13B) between the longitudinal axis of each soil sensor in each of the two adjacent sets is smaller than 10% of the predetermined horizontal distance value (e.g., horizontal distance 430) between the two adjacent sets (e.g., adjacent sets 420a, 420c, as shown in FIG. 13B).

Method 700 can include transmitting 720, by each soil sensor in the at least one set, signal to the target soil. Method 700 can include measuring 730, by each soil sensor in the at least one set, signals in the target soil.

Method 700 can include receiving 740, by each soil sensor in the at least one set (e.g., soil sensors 500-2 in set 420a, as shown in FIG. 13A), signals from an adjacent soil sensor positioned therebelow in that set (e.g., soil sensors 500-3 in set 420a). Method 700 can include transmitting 750, by each soil sensor in the at least one set (e.g., soil sensors 500-2 in set 420a), signals to an adjacent soil sensor positioned thereabove in that set (e.g., soil sensors 500-1 in set 420a). Method 700 can include transmitting 760, by a topmost soil sensor in the at least one set (e.g., soil sensors 500-1 in set 420a), signals to a base station (e.g., base station 410, as shown in FIG. 13A).

In some embodiments, the signals include information regarding at least one of: a volumetric water content (VWC), a temperature, a pH, a pressure, a salinity, a level of minerals of the target soil and any combination thereof. In some embodiments, the signals being transmitted by each soil sensor in the at least one set (e.g., soil sensor 500-2 in set 420a, as shown in FIG. 13A) to the adjacent soil sensor positioned thereabove in that set (e.g., soil sensor 500-1 in set 420a) include the information received from all soil sensors positioned therebelow in that set and the information measured by that soil sensor (e.g., soil sensors 500-3, 500-2 in set 420a). In some embodiments, each soil sensor in the at least one set transmits signals at different time sequences, different frequencies, with different spreading codes and any combination thereof to avoid an interference between the signals in that set and in two adjacent sets of soil sensors.

Method 700 can include determining 770, based on the received signals in the base station, the profile of properties of the target soil. In some embodiments, each of the soil sensors in each of the sets (e.g., soil sensor 500-2 in set 420a) compares received signal quality information from an adjacent soil sensor positioned therebelow in that set (e.g., soil sensor 500-3 in set 420a) with expected quality information to determine a change in the signal quality. In some embodiments, the profile properties below the surface of the target soil are determined based on the change in signal quality (e.g., between the quality of received signal and the expected quality of the signal) between the adjacent soil sensors.

In some embodiments, the transmitted and received signals are selected from a group comprising: electromagnetic signals, radiofrequency signals, ultrasonic signals or any combination thereof.

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

It is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein is for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. An underground soil sensors system, the system comprising:
a base station comprising at least one antenna; and
at least one set of soil sensors, each soil sensor in the at least one set is positioned at a predetermined vertical distance below a surface of a target soil,
wherein each soil sensor in the at least one set to transmit signals to an adjacent soil sensor positioned thereabove in that set and to receive signals from an adjacent soil sensor positioned therebelow in that set, and wherein a topmost soil sensor in the at least one set to transmit signals to the at least one antenna of the base station.

2. The underground soil sensors system of claim 1, wherein the transmitted and received signals are selected from a group comprising: electromagnetic (EM) signals, radiofrequency (RF) signals, ultrasonic (US) signals, infrared (IR) signals and near infrared (NIR) signals.

3. The underground soil sensors system of claim 1, wherein the signals comprising information regarding at least one of: a volumetric water content (VWC), a temperature, a pH, a pressure, a salinity, a level of minerals of the target soil and any combination thereof.

4. The underground soil sensors system of claim 1, wherein the signals being transmitted by each soil sensor in the at least one set to the adjacent soil sensor positioned thereabove in that set comprising the information received from that soil sensor and from all soil sensors positioned therebelow in that set.

5. The underground soil sensors system of claim 1, wherein each soil sensor in the at least one set comprises at least two soil probes positioned along the longitudinal axis of that soil sensor such that a vertical distance between two adjacent soil probes of that soil sensor having a first length value.

6. The underground soil sensors system of claim 5, wherein each soil probe of each soil sensor in the at least one set to transmit signals to an adjacent soil probe positioned thereabove in that soil sensor and to receive signals from an adjacent soil probe positioned therebelow in that soil sensor.

7. The underground soil sensors system of claim 6, wherein a topmost soil probe of each soil sensor in the at least one set to transmit signals to a bottommost soil probe of the adjacent soil sensor positioned thereabove in that set and wherein a bottommost soil probe of that soil sensor to receive signals from a topmost soil probe of the adjacent soil sensor positioned therebelow in that set.

8. The underground soil sensors system of claim 5, wherein a vertical distance between a bottommost soil probe of each soil sensor in the at least one set and a topmost soil probe of the adjacent soil sensor positioned therebelow in that set having the first length value.

9. The underground soil sensor system of claim 1, wherein a longitudinal axis of each soil sensor in the at least one set is substantially aligned along a longitudinal axis of the topmost soil sensor in that set, and wherein the longitudinal axis of the topmost soil sensor in the at least one set is substantially parallel to gravitational force.

10. The underground soil sensors system of claim 1, wherein a horizontal distance between two adjacent sets of soil sensors having a predetermined value.

11. The underground soil sensors system of claim 10, wherein the horizontal distance value is predetermined to avoid interference between transmissions of signals in the two adjacent sets.

12. The underground soil sensors system of claim 11, wherein a horizontal offset between the longitudinal axis of each soil sensor in each of the two adjacent sets is smaller than 10% of the predetermined horizontal distance value between the two adjacent sets.

13. The underground soil sensors system of claim 1, wherein each soil sensor in the at least one set to transmit signals at different time sequences, different frequencies, with different spreading codes and any combination thereof to avoid an interference between the signals in that set and in two adjacent sets of soil sensors.

14. The underground soil sensors system of claim 1, wherein each soil sensor in the at least one set comprising:
a rotatably anchorable portion to be rotatably anchored in a soil;
at least one soil probe mounted onto the rotatably anchorable portion; and
a communicator for communicating at least one output of the at least one soil probe to a location remote from the at least one soil sensor assembly.

15. The underground soil sensors system of claim 1, wherein each soil sensor in the at least one set is at least one of: a volumetric water content (VWC) sensor, a temperature sensor, a pH sensor, a pressure sensor, a salinity sensor, a sensor for determining level of minerals in a target soil and any combination thereof.

16. The underground soil sensors system of claim 1, wherein at least a portion of the at least one of the soil sensors in the at least one of the sets is positioned above the surface of the target soil.

17. A method of determining a profile of properties of a target soil, the method comprising:
installing at least one set of soil sensors such that each soil sensor in the at least one set is positioned at a predetermined depth below the surface of the target soil;
transmitting, by each soil sensor in the at least one set, signal to the target soil;
measuring, by each soil sensor in the at least one set, signals in the target soil;
receiving, by each soil sensor in the at least one set, signals from an adjacent soil sensor positioned therebelow in that set;
transmitting, by each soil sensor in the at least one set, signals to an adjacent soil sensor positioned thereabove in that set;
transmitting, by a topmost soil sensor in the at least one set, signals to a base station; and
determining, based on the received signals in the base station, the profile of properties of the target soil.

18. The method of claim 17, wherein the signals comprising information regarding at least one of: a volumetric water content (VWC), a temperature, a pH, a pressure, a salinity, a level of minerals of the target soil and any combination thereof.

19. The method of claim 18, wherein the signals being transmitted by each soil sensor in the at least one set to the adjacent soil sensor positioned thereabove in that set comprising the information received from all soil sensors positioned therebelow in that set and the information measured by that soil sensor.

20. The method of claim 17, wherein a longitudinal axis of each soil sensor in the at least one set is substantially aligned along a longitudinal axis of the topmost soil sensor in that set, and wherein the longitudinal axis of the topmost soil sensor in the at least one set is substantially parallel to gravitational force.

21. The method of claim 17, wherein a horizontal distance between two adjacent sets of soil sensors having a predetermined value.

22. The method of claim 21, wherein the horizontal distance value is predetermined to avoid interference between transmissions of signals in the two adjacent sets.

23. The method of claim 21, wherein a horizontal offset between the longitudinal axis of each soil sensor in each of the two adjacent sets is smaller than 10% of the predetermined horizontal distance value between the two adjacent sets.

24. The method of claim 17, wherein each soil sensor in the at least one set transmits signals at different time sequences, different frequencies, with different spreading codes and any combination thereof to avoid an interference between the signals in that set and in two adjacent sets of soil sensors.

25. The method of claim 17, wherein each of the soil sensors in each of the sets compares received signal quality information from an adjacent soil sensor positioned therebelow in that set with expected quality information to determine a change in the signal quality.

26. The method of claim 25, wherein the profile properties below the surface of the target soil are determined based on the change in signal quality between the adjacent soil sensors.

27. The method of claim 17, wherein the transmitted and received signals are selected from a group comprising: electromagnetic signals, radiofrequency signals, ultrasonic signals or any combination thereof.
